(19) United States
(12) Patent Application Publication
(10) Pub. No.: US 2024/0335297 A1
(43) Pub. Date: Oct. 10, 2024

(54) EXPANDABLE INTERVERTEBRAL FUSION CAGE

(71) Applicant: Medos International Sarl, Le Locle (CH)

(72) Inventor: Jonathan Melchor, Providence, RI (US)

(21) Appl. No.: 18/746,506

(22) Filed: Jun. 18, 2024

Related U.S. Application Data
(63) Continuation of application No. 18/362,154, filed on Jul. 31, 2023, now Pat. No. 12,023,258.
Application No. 18/362,154 is a continuation of application No. 17/223,504, filed on Apr. 6, 2021, now Pat. No. 11,752,009.

(51) Int. Cl. A61F 2/44; A61F 2/30

(52) U.S. Cl. CPC A61F 2/4465; A61F 2002/30266; A61F 2002/30289; A61F 2002/30331; A61F 2002/30405; A61F 2002/30556; A61F 2002/30579

(57) ABSTRACT

An intervertebral implant includes a first endplate member and a second endplate member, and a distal wedge member and a proximal wedge member that couple the first and second endplate members together. The distal wedge member is configured to move in an expansion direction that causes the fusion cage to move from a contracted position to an expanded position.

### CROSS-REFERENCE TO RELATED APPLICATIONS

This is a continuation of U.S. patent application Ser. No. 18/362,154 filed Jul. 31, 2023, which is a continuation of U.S. patent application Ser. No. 17/223,504 filed Apr. 6, 2021, the disclosure of which is hereby incorporated by reference as if set forth in its entirety herein for all purposes.

### TECHNICAL FIELD

The present invention relates to an expandable intervertebral implant.

### BACKGROUND

The human spine is comprised of a series of vertebral bodies separated by intervertebral discs. The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus within its cushioning qualities. These nucleus pulposus cells may also secrete small amounts of cytokines such as interleukin-1.beta. and TNF-.alpha. as well as matrix metalloproteinases (“MMPs”). These cytokines and MMPs help regulate the metabolism of the nucleus pulposus cells.

In some instances of degenerative disc disease (DDD), gradual degeneration of the intervertebral disc is caused by mechanical instabilities in other portions of the spine. In these instances, increased loads and pressures on the nucleus pulposus cause the cells within the disc (or invading macrophages) to emit larger than normal amounts of the above-mentioned cytokines. In other instances of DDD, genetic factors or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of proinflammatory cytokines and/or MMPs that may cause nerve irritation and pain.

As DDD progresses, toxic levels of the cytokines and MMPs present in the nucleus pulposus begin to degrade the extracellular matrix. In particular, the MMPs (as mediated by the cytokines) begin cleaving the water-retaining portions of the proteoglycans, thereby reducing their water-retaining capabilities. This degradation leads to a less flexible nucleus pulposus, and so changes the loading pattern within the disc, thereby possibly causing delamination of the annulus fibrosis. These changes cause more mechanical instability, thereby causing the cells to emit even more cytokines, typically thereby upregulating MMPs. As this destructive cascade continues and DDD further progresses, the disc begins to bulge (“a herniated disc”), and then ultimately ruptures, causing the nucleus pulposus to contact the spinal cord and produce pain.

One proposed method of managing these problems is to remove the problematic disc and replace it with a porous device that restores disc height and allows for bone growth therethrough for the fusion of the adjacent vertebrae. These devices are commonly called “fusion devices”.

One proposed method of managing these problems is to remove the problematic disc and replace it with a device that restores disc height and allows for bone growth between the adjacent vertebrae. These devices are commonly called fusion devices, or “interbody fusion devices”. Current spinal fusion procedures include transforaminal lumbar interbody fusion (TLIF), posterior lumbar interbody fusion (PLIF), anterior to posas (ATP), extraforaminal lumbar interbody fusion (ELIF), and lateral lumbar interbody fusion (LLIF) procedures. While interbody fusion devices are known in the art, there continues to be a need for minimally invasive devices that stabilize the spinal segment and create an optimum space for spinal fusion.

### SUMMARY

In one aspect, an expandable intervertebral fusion cage is configured for insertion in an intervertebral space defined between a superior vertebral body and an inferior vertebral body. The fusion cage can include an upper endplate member defining an upper bone contacting surface configured to abut the superior vertebral body, and a lower endplate member defining a lower bone contacting surface configured to abut the inferior vertebral body. The upper and lower bone contacting surfaces can be opposite each other along a transverse direction. The fusion cage can further include a wedge member having at least one ramp surface, and a drive member that extends along a curved central axis. The wedge member can be supported by the drive member. The fusion cage can further include an actuator that is configured to cause the drive member to drive the wedge member to translate in a corresponding expansion direction along the curved central axis without relative rotation between the wedge member and the drive member, which causes the ramp surface of the wedge member to urge at least one of the upper and lower endplate members away from the other of the upper and lower endplate members along the transverse direction.

### BRIEF DESCRIPTION OF THE DRAWINGS

The foregoing summary, as well as the following detailed description of illustrative embodiments of an intervertebral fusion cage of the present application, will be better understood when read in conjunction with the appended drawings. For the purposes of illustrating the expandable fusion cage of the present application, there is shown in the drawings illustrative embodiments. It should be understood, however, that the application is not limited to the precise arrangements and instrumentalities shown. In the drawings:

FIG. 1 is a perspective view of an intervertebral fusion cage disposed in an intervertebral space in a contracted position;

FIG. 2 is a perspective view of the fusion cage illustrated in FIG. 1, but shown expanded from the contracted position to an expanded position;

FIG. 3 is an exploded perspective view of the fusion cage illustrated in FIG. 1, including upper and lower endplate members, distal and proximal wedge members, and a drive assembly;

FIG. 4A is a perspective view of the upper endplate member illustrated in FIG. 3;

FIG. 4B is another perspective view of the upper endplate member illustrated in FIG. 3;

FIG. 5A is a perspective view of the lower endplate member illustrated in FIG. 3;

FIG. 5B is another perspective view of the lower endplate member illustrated in FIG. 3;

FIG. 6A is a perspective view of the distal wedge member illustrated in FIG. 3;

FIG. 6B is another perspective view of the distal wedge member illustrated in FIG. 3;

FIG. 7A is a perspective view of the proximal wedge member illustrated in FIG. 3;

FIG. 7B is another perspective view of the proximal wedge member illustrated in FIG. 3;

FIG. 8A is a perspective view of the fusion cage illustrated in FIG. 3, shown in a contracted position;

FIG. 8B is a side elevation view of the fusion cage illustrated in FIG. 8A;

FIG. 8C is another side elevation view of the fusion cage illustrated in FIG. 8B;

FIG. 8D is a perspective view of the fusion cage illustrated in FIG. 8A, shown in an expanded position;

FIG. 9A is a sectional perspective view of the fusion cage illustrated in FIG. 8A;

FIG. 9B is a cross-sectional view of the fusion cage illustrated in FIG. 9A;

FIG. 10A is a sectional perspective view of the fusion cage similar to FIG. 9A, but showing the fusion cage in the expanded position;

FIG. 10B is a cross-sectional view of the fusion cage illustrated in FIG. 10A;

FIG. 11A is a perspective view of a drive member of the drive assembly illustrated in FIG. 3; and

FIG. 11B is a top plan view of the drive member illustrated in FIG. 11A.

### DETAILED DESCRIPTION OF ILLUSTRATIVE EMBODIMENTS

Certain terminology is used in the following description for convenience only and is not limiting. The “lower” and “upper” designate directions in the drawings to which reference is made. The words, “anterior”, “posterior”, “superior,” “inferior,” “medial,” “lateral,” and related words and/or phrases are used to designate various positions and orientations in the human body, but also applies to the fusion cage when disposed outside the human body. The terminology includes the above-listed words, derivatives thereof and words of similar import.

Unless otherwise indicated, the terms “substantially,” “generally,” and “approximately” along with derivatives thereof and words of similar import as used herein with respect to dimensions, values, shapes, directions, and other parameters can include the stated dimensions, values, shapes, directions, and other parameters and up to plus or minus 10% of the stated dimensions, values, shapes, directions, and other parameters, such as up to plus or minus 9% of the stated dimensions, values, shapes, directions, and other parameters, such as up to plus or minus 8% of the stated dimensions, values, shapes, directions, and other parameters, such as up to plus or minus 7% of the stated dimensions, values, shapes, directions, and other parameters, such as up to plus or minus 6% of the stated dimensions, values, shapes, directions, and other parameters, such as up to plus or minus 5% of the stated dimensions, values, shapes, directions, and other parameters, such as up to plus or minus 4% of the stated dimensions, values, shapes, directions, and other parameters, such as up to plus or minus 3% of the stated dimensions, values, shapes, directions, and other parameters, such as up to plus or minus 2% of the stated dimensions, values, shapes, directions, and other parameters, such as up to plus or minus 1% of the stated dimensions, values, shapes, directions, and other parameters.

Method steps and apparatus described or referenced herein will sometimes be described in singular form for clarity. However, it should be appreciated that as used herein, the singular term “a” or “the” with respect to an apparatus or method step can include the plural apparatus or method steps. Conversely, the plural term as used herein with respect to apparatus or method steps can include the singular “a” or “the.” Thus, it should be appreciated that the use herein of the singular term “a” or “the” and the use herein of the plural term can equally apply to “at least one” unless otherwise indicated.

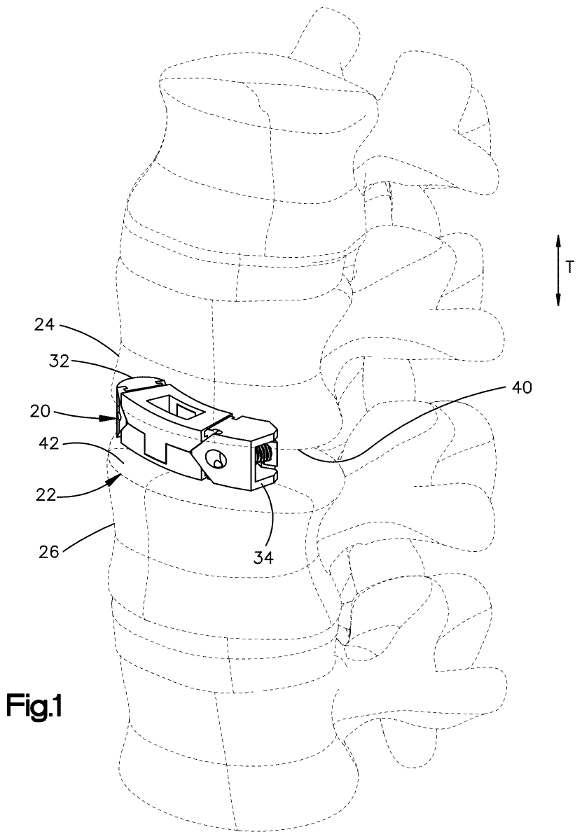

Referring to FIG. 1, an intervertebral implant or fusion cage 20 can be inserted into an intervertebral space 22 in an initial or contracted position. The intervertebral space 22 can be disposed along the lumbar region of the spine, but it is envisioned that the fusion cage can be positioned at any alternative location of the spine, such as the thoracic region or cervical region of the spine. The intervertebral space 22 can be defined by a first or superior vertebral body 24 and a second or inferior vertebral body 26. The superior vertebral body 24 defines a superior vertebral surface 40, and the adjacent inferior vertebral body 26 defines an inferior vertebral surface 42. The superior vertebral surface 40 and the inferior vertebral surface 42 can face each other so as to define the intervertebral space 22. The vertebral bodies 24 and 26 can be anatomically adjacent vertebral bodies. The intervertebral space 22 of FIG. 1 is illustrated after a discectomy has been performed, whereby the disc material has been removed or at least partially removed to prepare the intervertebral space 22 to receive the fusion cage 20.

The fusion cage 20 can be inserted into the intervertebral space 22 along any suitable approach as desired, such as a transforaminal approach during a transforaminal lumbar interbody fusion (TLIF) procedure. The fusion cage 20 thus defines a distal end 32 which can also define a leading end of the fusion cage 20 with respect to insertion into the intervertebral space 22, and a proximal end 34 that can also define a trailing end of the fusion cage 20 that is opposite the distal end 32. As used herein, the term “proximal” and derivatives thereof refer to a direction from the distal end 32 toward the proximal end 34. As used herein, the term “distal” and derivatives thereof refer to a direction from the proximal end 34 toward the distal end 32.

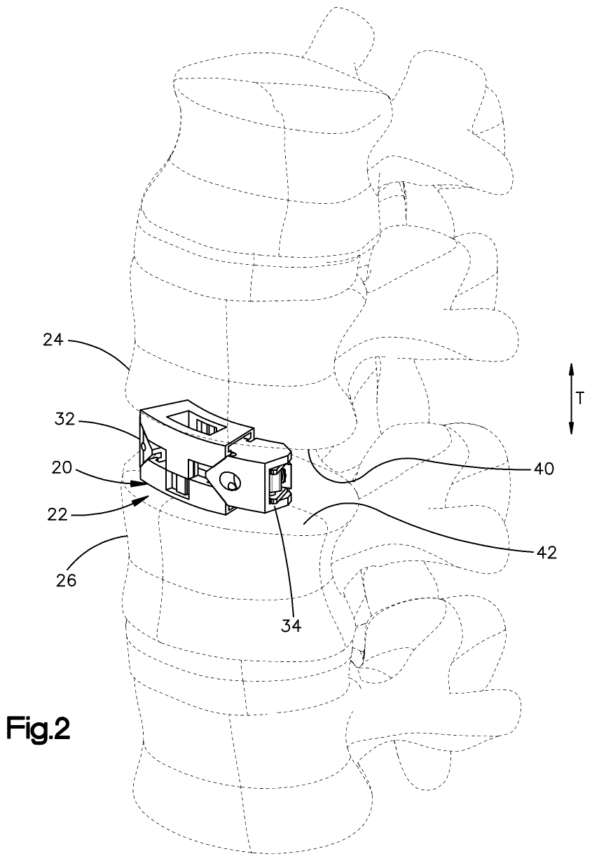

Referring now to FIG. 2, once the fusion cage 20 has been inserted into the intervertebral space 22, the fusion cage 20 can be expanded from the first or contracted position to a second or expanded position. The fusion cage 20 achieves a height in the expanded position that is greater than the height of the fusion cage 20 in the contracted position along a transverse direction T. As a result, the fusion cage 20 can achieve height restoration of the intervertebral space 22.

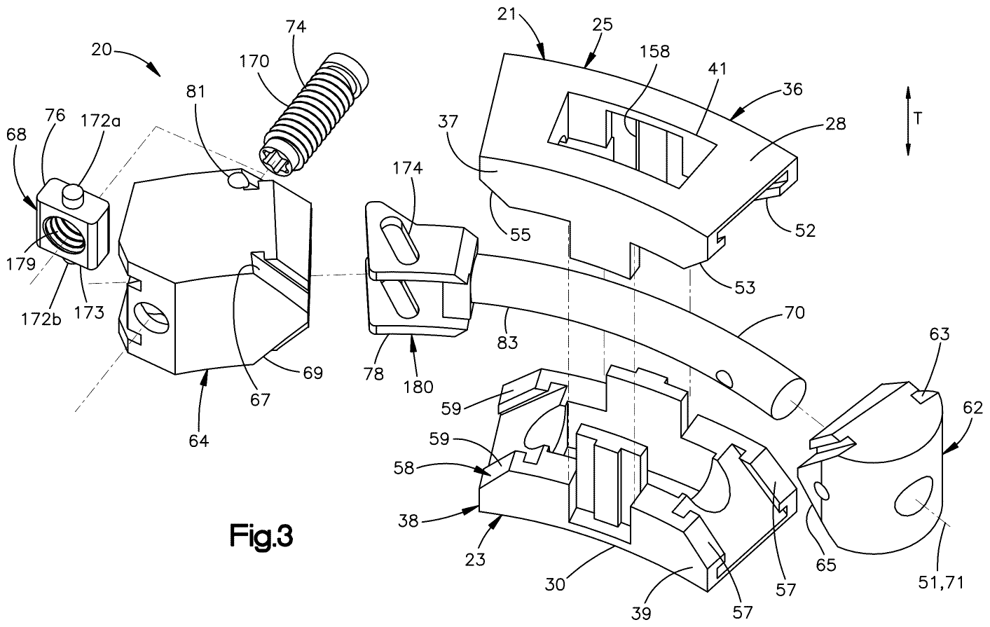

Referring also to FIG. 3, the expandable fusion cage 20 defines a cage body 21 having a first or upper endplate member 36 and a second or lower endplate member 38 opposite the upper endplate member along the transverse direction T. The cage body 21, and thus the cage 20 including each of the upper and lower endplate members 36 and 38, defines a first side 23 and a second side 25 opposite the first side 23. The endplate members 36 can be formed of polyether ether ketone (PEEK) or any other suitable biocompatible polymeric material. Alternatively, the upper and lower endplate members 36 and 38 can be made of any suitable biocompatible metal such as a titanium-aluminum-niobium (TAN) alloy. It should be appreciated that the any suitable alternative material can be used to form the endplate members 36 and 38 as desired.

The upper endplate member 36 includes an upper plate portion 37 that defines an upper bone contacting surface 28 that is configured to abut or grip the superior vertebral surface 40, and an upper inner surface 29 that is opposite the upper bone contacting surface 28 along the transverse direction. The lower endplate member 38 includes a lower plate portion 39 that defines a lower bone contacting surface 30 that is configured to abut or grip the inferior vertebral surface 42, and a lower inner surface 31 that is opposite the lower bone contacting surface 30 along the transverse direction T. The bone contacting surfaces 28 and 30 can be opposite each other along the transverse direction T. The term “up” and “above” and derivatives thereof refer to a direction from the lower bone contacting surface 30 toward the upper bone contacting surface 28. The term “down” and “below” derivatives thereof refer to a direction from the upper bone contacting surface 28 toward the lower bone contacting surface 30.

Each of the upper and lower bone contacting surfaces 28 and 30 can be convex or partially convex, for instance, one portion of the surface is convex while another portion can be substantially planar. Alternatively, each of the upper and lower bone contacting surfaces 28 and 30 can be substantially planar along their respective entireties. While the upper and lower bone contacting surfaces 28 and 30 can be smooth in some examples, it is recognized that the upper and lower endplate members 36 and 38 can include any suitable texture that extends from the upper and lower bone contacting surfaces 28 and 30, such as teeth, spikes, ridges, cones, barbs, indentations, or knurls, which are configured to grip the superior and inferior vertebral bodies 24 and 26, respectively, to resist migration of the fusion cage 20 in the intervertebral space 22.

The cage body 21 can be a curved cage body. In one example, the cage body can define a banana shape in a plane that is perpendicular to the transverse direction T. Accordingly, the cage body 21, and thus, the fusion cage 20, can extend along a curved central axis 51 that extends from the proximal end 34 to the distal end 32. In particular, the central axis 51 can be curved in a plane that is perpendicular to the transverse direction T. The curved central axis 51 can define a constant curvature from the distal end 32 to the proximal end 34. The fusion cage 20 is designed and configured to be inserted into an intervertebral space in a direction from the trailing end 34 toward the insertion end 32, also referred to herein as an insertion direction. The insertion direction can also be oriented along the curved central axis 51, or any suitable alternative direction as desired. The proximal and distal directions can be oriented along the central axis 51, or along a direction parallel to the central axis 51. The first and second sides 23 and 25 of the cage body 21 can be disposed on opposite sides of the central axis 51. In particular, the first side 23 of the cage body 21 can be concave, and the second side 25 of the cage body 21 can be convex.

As described above, the fusion cage 20 is configured to be expanded from a collapsed position having first height to an expanded position having second height greater than the first height. The first and second height can be measured along the transverse direction T from the upper bone contacting surface 28 to the lower bone contacting surface 30. In this regard, the upper endplate member 36 can define at least one upper ramp surface 52 that is sloped with respect to a plane that is perpendicular to the transverse direction. Alternatively or additionally, the lower endplate member 38 can define at least one lower ramp surface 58 that is sloped with respect to a plane that is perpendicular to the transverse direction. The fusion cage 20 can further include at least one wedge member configured to move in an expansion direction along a curved path that can be defined by the curved central axis 51. The curved path can extend along the curved central axis 51 or can be defined by a respective curved line that extends parallel to the curved central axis 51. Movement of the at least one wedge member in the expansion direction causes a ramp surface of the wedge member to urge at least one of the upper and lower endplate members 36 and 38 away from the other of the upper and lower endplate members 36 and 38 along the transverse direction T, thereby expanding the fusion cage 20. In particular, relative translation between the at least one ramp surface of the at least one wedge member and at least one or both of the at least one upper ramp surface 52 and the at least one lower ramp surface 58 can cause the fusion cage 20 to expand.

In one example, the at least one upper ramp surface 52 of the upper endplate member 36 can include at least one first or distal upper ramp surface 53 and at least one second or proximal upper ramp surface 55 opposite the first ramp surface 53 along the central axis 51. The at least one distal upper ramp surface 53 can be spaced from the proximal upper ramp surface 55 in the distal direction along the central axis 51. The at least one lower ramp surface 58 of the lower endplate member 38 can include at least one first or distal lower ramp surface 57 and at least one second or proximal lower ramp surface 59 opposite the first lower ramp surface 57 along the central axis 51. The at least one distal lower ramp surface 57 can be spaced from the proximal lower ramp surface 59 in the distal direction. The at least one wedge member can include a first or distal wedge member 62 and a second or proximal wedge member 64 that is opposite the first wedge member 62. The first wedge member 62 can include at least one upper distal wedge ramp surface 63 and at least one lower distal wedge ramp surface 65. The second wedge member 64 can similarly include at least one upper proximal wedge ramp surface 67 and at least one lower proximal wedge ramp surface 69.

The at least one first upper ramp surface 53 of the upper endplate member 36 can be sloped upward, or toward the upper bone contacting surface 28, as it extends in the distal direction. The at least one second upper ramp surface 55 of the upper endplate member 36 can be sloped upward as it extends in the proximal direction. The at least one first lower ramp surface 57 of the lower endplate member 38 can be sloped downward, or toward the lower bone contacting surface 30, as it extends in the distal direction. The at least one second lower ramp surface 59 of the lower endplate member 38 can be sloped downward as it extends in the proximal direction. The at least one upper distal wedge ramp surface 63 of the first wedge member 62 can be sloped upward as it extends in the distal direction. The at least one lower distal wedge ramp surface 65 of the first wedge member 62 can be sloped downward as it extends in the distal direction. The at least one upper proximal wedge ramp surface 67 of the second wedge member 64 can be sloped upward as it extends in the proximal direction. The at least one lower proximal wedge ramp surface 69 of the second wedge member 64 can be sloped downward as it extends in the proximal direction.

The first wedge member 62 is movable along an expansion direction, which causes (i) the at least one upper distal wedge ramp surface 63 and the at least one lower distal wedge ramp surface 65 of the first wedge member 62 to ride along the at least one distal upper ramp surface 53 of the upper endplate member 36 the at least one distal lower ramp surface 57 of the lower endplate member 38, respectively, and (ii) the at least one proximal upper ramp surface 55 of the upper endplate member 36 and the at least one proximal lower ramp surface 59 of the lower endplate member 38 to ride along the at least one upper proximal wedge ramp surface 67 and at least one lower proximal wedge ramp surface 69, respectively, of the second wedge member 64, thereby causing the upper and lower endplate members 36 and 38 to move away from each other along the transverse direction T.

The expansion direction of the first wedge member 62 can be along the curved path defined by the central axis 51. Further, the expansion direction of the first wedge member 62 can be toward the second wedge member 64. Alternatively, the fusion cage 20 can be configured such that the expansion direction of the first wedge member 62 can be away from the second wedge member 64. For instance, the ramp surfaces described herein can be oppositely sloped, such that movement of the first wedge member 62 away from the second wedge member 64 each other can cause the fusion cage 20 to expand along the transverse direction T.

With continuing reference to FIG. 3, the fusion cage 20 can include a drive assembly 68 that is configured to drive the first wedge member 62 to move selectively in the expansion direction that causes the height of the fusion cage 20 to increase, and in a contraction direction that causes the height of the fusion cage 20 to decrease. The drive assembly 68 can include a drive member 70 that is disposed between the upper and lower endplate members 36 and 38, and is coupled to the first wedge member 62. The drive member 70 can include a shaft 83 that is elongate along a respective central axis 71, and a coupling 78 that extends from the shaft 83. In one example, the coupling 78 can extend proximally from the shaft 83. The central axis 71 of the drive member 70 can be curved in a plane that is oriented perpendicular to the transverse direction T. For instance, the central axis 71 of the drive member 70 can define the same curvature as the central axis 51 of the fusion cage 20, and thus can extend along the same curved path as the central axis 51. Accordingly, the central axes 51 and 71 can be parallel with each other. Thus, the central axes 71 and 51 can be said to extend along a common curved path In one example, the central axes 51 and 71 can extend along respective circular paths. In some examples, the central axes 51 and 71 can be concentric. In one example, the central axes 51 and 71 can be coincident with each other. The drive assembly 68 further includes an actuator 74 and a traveler 76 that is supported by the actuator 74. The actuator 74, in turn, can be supported by the second wedge member 64. As will be described in more detail below, the actuator 74 is configured to move in an actuation direction that causes the traveler 76 to travel along the actuator 74, which in turn causes the traveler 76 to move the coupling 78, and thus the drive member 70, in a direction that causes the first wedge member 62 to move in the expansion direction. The actuator 74 is configured to move in a second direction opposite the actuation direction that causes the traveler 76 to travel along the actuator 74, which in turn causes the traveler 76 to move the coupling 78, and thus the drive member 70, in a direction that causes the first wedge member 62 to move in the contraction direction. The second wedge member 64 can define an opening 81 that receives a pin to further capture the coupling 78, and thus the drive member 70.

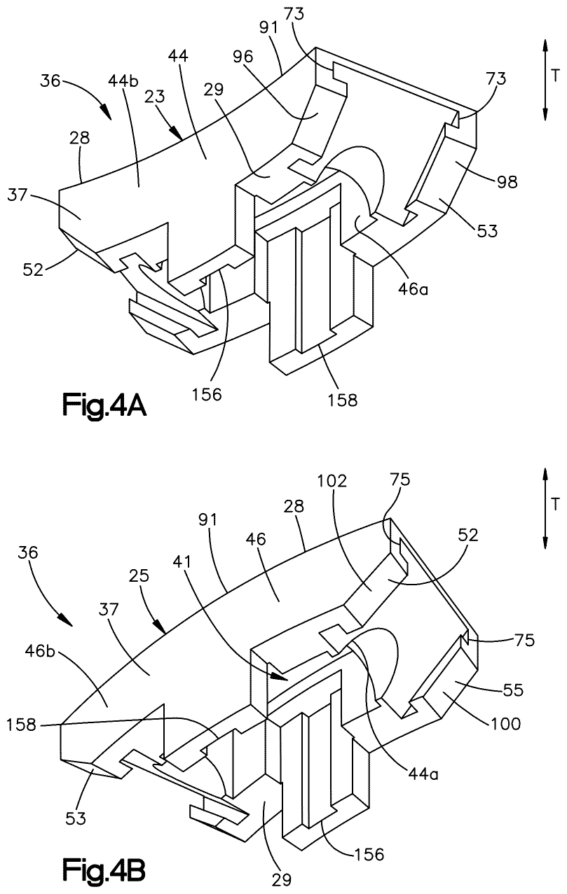

The upper endplate member 36 will now be described in more detail with reference to FIGS. 3-4B. The upper endplate member 36 can include the upper plate portion 37 having an outer surface 91 that defines the upper bone contacting surface 28, and the upper inner surface 29 opposite the outer surface 91. The upper endplate member 36 can further include at least one upper bone graft aperture 41 that extends through the upper plate portion 37 from the upper bone contacting surface 28 to the upper inner surface 29. The bone graft aperture 41 is configured to receive bone graft material to enhance fusion of the upper endplate member 36 with the superior vertebral body 24.

The upper endplate member 36 defines a first upper side wall 44 and a second upper side wall 46 that is opposite the first upper side wall 44. The first and second upper side walls 44 and 46 extend down from the upper plate portion 37, and in particular from the upper inner surface 29. The first upper side wall 44 is disposed at the first side 23 of the cage body 21. The second upper side wall 46 is disposed at the second side 25 of the cage body 21. The first and second upper side walls 44 and 46 define respective inner surfaces 44a and 46a that generally face each other, and respective outer surfaces 44b and 46b opposite the respective inner surfaces. In one example, the outer surface 44b of the first upper side wall 44 can be concave. The inner surface 44a of the first upper side wall 44 can be convex. Further, the inner surface 44a of the first upper side wall 44 can be substantially parallel with the outer surface 44b of the first upper side wall 44. The outer surface 46b of the second upper side wall 46 can be convex. The inner surfaces 46a of the upper side wall 46 can be concave. Further, the inner surface 46a of the upper side wall 46 can be substantially parallel with the outer surface 46b of the second upper side wall 46.

As described above, the upper endplate member 36 can include at least one first or distal upper ramp surface 53 and at least one second or proximal upper ramp surface 55 opposite the first ramp surface 53 along the central axis 51. In particular, the at least one first upper ramp surface 53 is spaced from the at least one second upper ramp surface 55 in the distal direction along the central axis 51. The at least one first upper ramp surface 53 can include first and second upper distal ramp surfaces 96 and 98, respectively, and the at least one second upper ramp surface 55 can include third and fourth upper ramp surfaces 100 and 102, respectively. The ramp surfaces 53 and 55 can be mirror images of each other. The first and second ramp surfaces 96 and 98 can be referred to as first and second upper distal ramp surfaces, respectively, of the upper endplate member 36. The third and fourth ramp surfaces 100 and 102 can be referred to as first and second upper proximal ramp surfaces, respectively, of the upper endplate member 36.

The first ramp surface 96 of the upper endplate member 36 can be disposed at the first side 23 of the fusion cage 20, and the second ramp surface 98 can be disposed at the second side 25 of the fusion cage 20. The first and second ramp surfaces 96 and 98 of the upper endplate member 36 can be spaced from each other, and opposite each other with respect to the central axis 51. Thus, the first and second ramp surfaces 96 and 98 can be disposed on opposite sides of the central axis 51. The helical paths of the first and second ramp surfaces 96 and 98 can be defined by a common helical pitch, but defined by different helical radii. For instance, the helical paths of the first and second ramp surfaces 96 and 98 can be defined by a single swept helix. The third ramp surface 100 of the upper endplate member 36 can be disposed at the first side 23 of the fusion cage 20, and the fourth ramp surface 102 can disposed at the second side 25 of the fusion cage 20. The third and fourth ramp surfaces 100 and 102 of the upper endplate member 36 can be spaced from each other and opposite each other with respect to the central axis 51. Thus, the third and fourth ramp surfaces 100 and 102 can be disposed on opposite sides of the central axis 51. The helical paths of the third and fourth ramp surfaces 100 and 102 can be defined by a common helical pitch, but defined by different helical radii. For instance, the helical paths of the third and fourth ramp surfaces 100 and 102 can be defined by a single swept helix. The first ramp surface 96 and the third ramp surface 100 can be aligned with each other along the curved path defined by the central axis 51, and the second ramp surface 98 and the fourth ramp surface 102 can be aligned with each other along the curved path defined by the central axis 51.

Each of the first, second, third, and fourth ramp surfaces 96-102 of the upper endplate member 36 can extend along a respective nonlinear path. Otherwise stated, each of the first, second, third, and fourth ramp surfaces 96-102 of the upper endplate member 36 can be nonplanar. In one example, the first and second ramp surfaces 96 and 98 of the upper endplate member 36 and the third and fourth ramp surfaces 100 and 102 of the upper endplate member 36 can extend along respective twisted paths. For instance, the first and second ramp surfaces 96 and 98 of the upper endplate member 36 can extend along respective helical paths. The helical paths of the first and second ramp surfaces 96 and 98 can be defined by a common helical pitch, but defined by different helical radii. For instance, the helical paths of the first and second ramp surfaces 96 and 98 can be defined by a single swept helix. Similarly, the third and fourth ramp surfaces 100 and 102 of the upper endplate member 36 can extend along respective helical paths. The helical paths of the third and fourth ramp surfaces 100 and 102 can be defined by the same helical pitch, but defined by different helical radii. For instance, the helical paths of the third and fourth ramp surfaces 100 and 102 can be defined by a single swept helix.

While the first and second ramp surfaces 96 and 98 are separate ramp surfaces spaced from each other in one example, it is recognized that the first and second ramp surfaces 96 and 98 can alternatively be continuous with each other so as to define a single ramp surface as desired. Similarly, while the third and fourth ramp surfaces 100 and 102 are separate ramp surfaces spaced from each other in one example, it is recognized that the third and fourth ramp surfaces 100 and 102 can alternatively be continuous with each other so as to define a single ramp surface as desired.

The upper endplate member 36 can be interlinked with the first and second wedge members 62 and 64. In one example, the upper endplate member 36 can include an interlinking member that is configured to engage complementary interlinking members of the first and second wedge members 62 and 64, respectively. For instance, the interlinking member of the upper endplate member 36 can include first or distal slots 73 disposed adjacent the first and second ramp surfaces 96 and 98, and second or proximal slots 75 that are disposed adjacent the third and fourth ramp surfaces 100 and 102. The first and second slots 73 and 75 can be configured to receive the complementary interlinking members of the first and second wedge members 62 and 64, respectively. It is recognized, of course, that the interlinking member of the upper endplate member 36 can alternatively define projections that is received in recesses of the first and second wedge members 62 and 64, respectively.

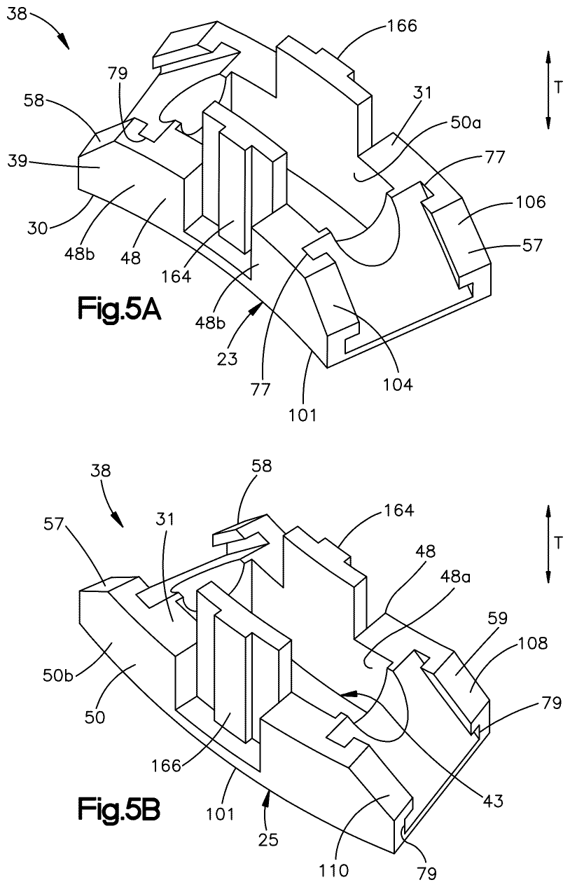

The lower endplate member 38 will now be described in more detail with reference to FIGS. 3 and 5A-5B. The lower endplate member 38 can include a lower plate portion 39 having an outer surface 101 that defines the lower bone contacting surface 30, and the lower inner surface 31 opposite the outer surface 101. The lower endplate member 38 can further include at least one lower bone graft aperture 43 that extends through the lower plate portion 39 from the lower bone contacting surface 30 to the lower inner surface 31. The lower bone graft aperture 43 is configured to receive bone graft material to enhance fusion of the lower endplate member 38 with the inferior vertebral body 26.

The lower endplate member 38 defines a first lower side wall 48 and a second lower side wall 50 that is opposite the first lower side wall 48. The first and second lower side walls 48 and 50 extend up from the lower plate portion 39, and in particular from the lower inner surface 31. The first lower side wall 48 is disposed at the first side 23 of the cage body 21. The first lower side wall 48 can be aligned with the first upper side wall 44 along the transverse direction T. The second lower side wall 50 is disposed at the second side 25 of the cage body 21. The second lower side wall 50 can be aligned with the second upper side wall 46 along the transverse direction T. The first and second lower side walls 48 and 50 define respective inner surfaces 48a and 50a that generally face each other, and respective outer surfaces 48b and 50b opposite the respective inner surfaces. In one example, the outer surface 48b of the first lower side wall 48 can be concave. The inner surface 48a of the first lower side wall 48 can be convex. Further, the inner surface 48a of the first lower side wall 48 can be substantially parallel with the outer surface 48b of the first lower side wall 48. The first lower side wall 48 and the first upper side wall 44 can be substantially aligned with each other along the transverse direction T. The outer surface 50b of the second lower side wall 50 can be convex. The inner surface 50a of the second lower side wall 50 can be concave. Further, the inner surface 50a of the second lower side wall 50 can be substantially parallel with the outer surface 50b of the second lower side wall 50.

As described above, the lower endplate member 38 can include at least one first or distal lower ramp surface 57 and at least one second or proximal lower ramp surface 59 opposite the first lower ramp surface 57 along the central axis 51. In particular, the at least one first lower ramp surface 57 is spaced from the at least one second lower ramp surface 59 in the distal direction along the central axis 51. The at least one distal lower ramp surface 57 can include first and second lower ramp surfaces 104 and 106, respectively, and the at least one proximal lower ramp surface 59 can include third and fourth lower ramp surfaces 108 and 110, respectively. The first and second lower ramp surfaces 104 and 106 can be referred to as first and second lower distal ramp surfaces, respectively, of the lower endplate member 38. The third and fourth ramp surfaces 108 and 110 can be referred to as first and second lower proximal ramp surfaces, respectively, of the lower endplate member 38.

The first ramp surface 104 of the lower endplate member 38 can be disposed at the first side 23 of the fusion cage 20, and the second ramp surface 106 can be disposed at the second side 25 of the fusion cage 20. The first and second ramp surfaces 104 and 106 of the lower endplate member 38 can be spaced from each other, and opposite each other with respect to the central axis 51. Thus, the first and second ramp surfaces 104 and 106 can be disposed on opposite sides of the central axis 51. The helical paths of the first and second ramp surfaces 104 and 106 can be defined by a common helical pitch, but defined by different helical radii. For instance, the helical paths of the first and second ramp surfaces 104 and 106 can be defined by a single swept helix. The third ramp surface 108 of the lower endplate member 38 can be disposed at the first side 23 of the fusion cage 20, and the fourth ramp surface 110 can disposed at the second side 25 of the fusion cage 20. The third and fourth ramp surfaces 108 and 110 of the lower endplate member 38 can be spaced from each other, and opposite each other with respect to the central axis 51. Thus, the third and fourth ramp surfaces 108 and 110 can be spaced from each other on opposite sides of the central axis 51. The helical paths of the third and fourth ramp surfaces 108 and 110 can be defined by a common helical pitch, but defined by different helical radii. For instance, the helical paths of the third and fourth ramp surfaces 108 and 110 can be defined by a single swept helix. The first ramp surface 104 and the third ramp surface 108 can be aligned with each other along the curved path defined by the central axis 51, and can be mirror images of each other. The second ramp surface 106 and the fourth ramp surface 110 can be aligned with each other along the curved path defined by the central axis 51, and can be mirror images of each other.

Each of the first, second, third, and fourth ramp surfaces 104-110 of the lower endplate member 38 can extend along a respective nonlinear path. Otherwise stated, each of the first, second, third, and fourth ramp surfaces 104-110 of the upper endplate member 36 can be nonplanar. In one example, the first and second ramp surfaces 104 and 106 of the lower endplate member 38 and the third and fourth ramp surfaces 108 and 110 of the lower endplate member 38 can extend along respective twisted paths. For instance, the first and second ramp surfaces 104 and 106 of the lower endplate member 38 can extend along respective helical paths. The helical paths of the first and second ramp surfaces 104 and 106 can be defined by a common helical pitch, but defined by different helical radii. For instance, the helical paths of the first and second ramp surfaces 104 and 106 can be defined by a single swept helix. Similarly, the third and fourth ramp surfaces 108 and 110 of the lower endplate member 38 can extend along respective helical paths. The helical paths of the third and fourth ramp surfaces 108 and 110 can be defined by the same helical pitch, but defined by different helical radii. For instance, the helical paths of the third and fourth ramp surfaces 108 and 110 can be defined by a single swept helix.

While the first and second lower ramp surfaces 104 and 106 are separate ramp surfaces spaced from each other in one example, it is recognized that the first and second lower ramp surfaces 104 and 106 can alternatively be continuous with each other so as to define a single ramp surface as desired. Similarly, while the third and fourth ramp surfaces 108 and 110 are separate ramp surfaces spaced from each other in one example, it is recognized that the third and fourth ramp surfaces 108 and 110 can alternatively be continuous with each other so as to define a single ramp surface as desired.

The lower endplate member 38 can be interlinked with the first and second wedge members 62 and 64. In one example, the lower endplate member 38 can include an interlinking member that is configured to engage complementary interlinking members of the first and second wedge members 62 and 64, respectively. For instance, the interlinking member of the lower endplate member 38 can include first or distal slots 77 disposed adjacent the first and second ramp surfaces 104 and 106, and second or proximal slots 79 that are disposed adjacent the third and fourth ramp surfaces 108 and 110. The first and second slots 77 and 79 can be configured to receive the complementary interlinking members of the first and second wedge members 62 and 64, respectively. It is recognized, of course, that the interlinking member of the lower endplate member 38 can alternatively define projections that is received in recesses of the first and second wedge members 62 and 64, respectively.

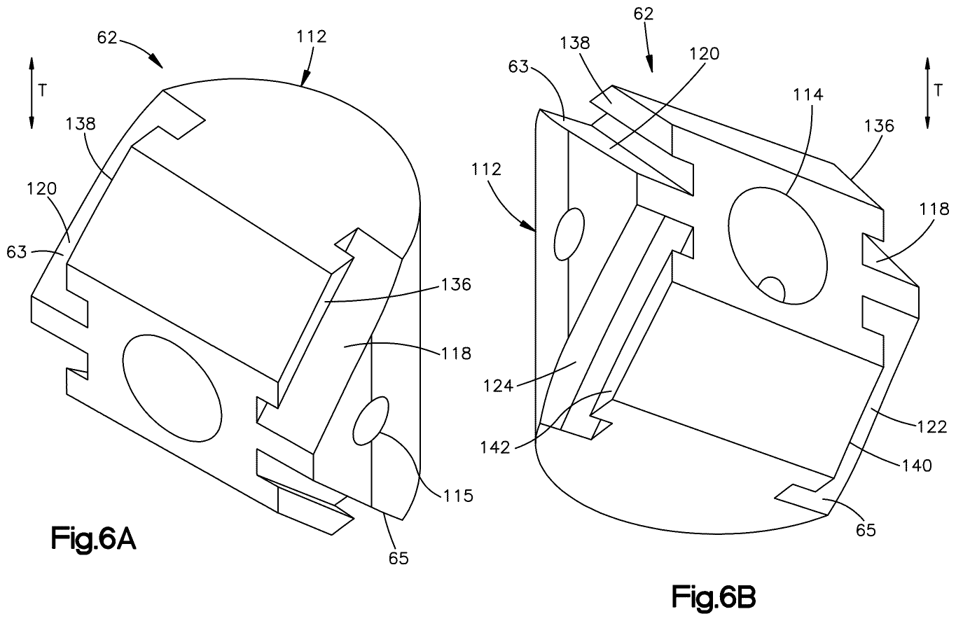

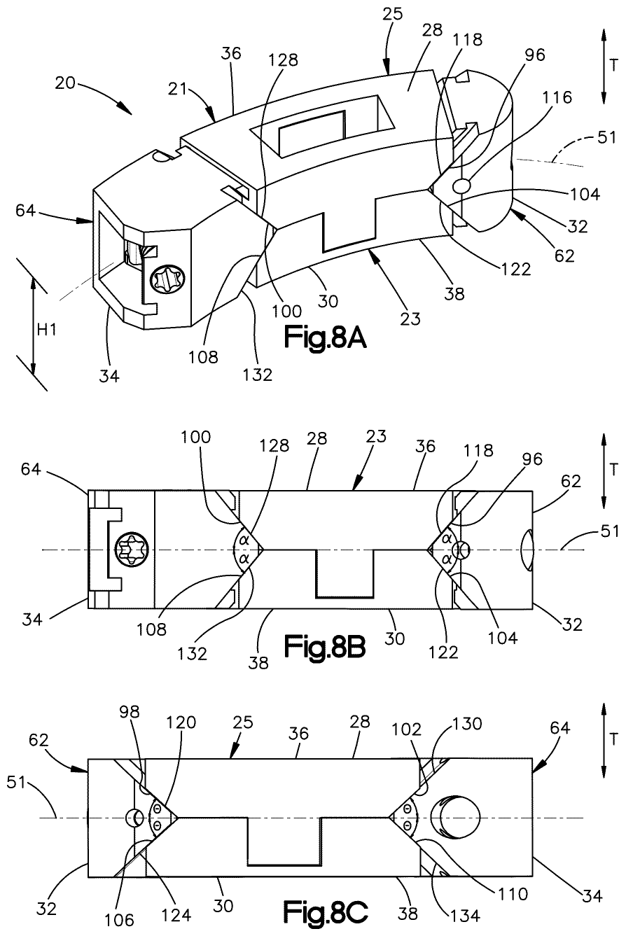

The first wedge member 62 will now be described with reference to FIGS. 3 and 6A-6B. The first wedge member 62 defines a first wedge body 112 that can be coupled to the drive member 70, such that the first wedge body 112 moves with the drive member 70. In one example, the first wedge member 62 can defines a first bore 114 that extends at least into or through the first wedge body 112. The first bore 114 can be sized to receive a distal end of the drive member 70. As described above, the drive member 70 can be curved. Thus, the first bore 114 can have a curvature that corresponds to the curvature of the drive member 70. The first wedge member 62 can further include a locking bore 115 that receives a locking pin 116 (see FIG. 8A) that is coupled to the drive member 70. For instance, the locking pin 116 can extend at least into or through the drive member 70. Thus, the locking pin 116 can couple the first wedge body 112, and thus the first wedge member 62, to the drive member 70. Accordingly, the first wedge member 62 can be both translatably and rotationally fixed to the drive member 70. Thus, translation and rotation of the first wedge member 62 with respect to the drive member 70 can be prevented. As a result, movement of the drive member 70 causes the first wedge member 62 to correspondingly move with the drive member 70. It should be appreciated that the first wedge member 62 can be coupled to the drive member 70 in any suitable alternative manner as desired. For instance, in some examples, the first wedge member 62 and the drive member 70 can define a single unitary structure.

With continuing reference to FIGS. 3 and 6A-6B, and as described above, the first wedge body 120, and thus the first wedge member 62, defines at least one upper distal wedge ramp surface 63 and at least one lower distal wedge ramp surface 65 opposite the upper distal ramp surface 63 along the transverse direction T. The at least one upper distal wedge ramp surface 63 of the first wedge member 62 can be sloped upward toward the upper bone contacting surface 28 as it extends in the distal direction. The at least one lower distal wedge ramp surface 65 of the first wedge member 62 can be sloped downward toward the lower bone contacting surface 30 as it extends in the distal direction.

The at least one upper distal wedge ramp surface 63 of the first or distal wedge member 62 can include first and second ramp surfaces 118 and 120. The at least one lower distal wedge ramp surface 65 of the first or distal wedge member 62 can include third and fourth ramp surfaces 122 and 124, respectively. The first and second ramp surfaces 118 and 120 can be referred to as first and second upper distal ramp surfaces, respectively, of the first wedge member 62. The third and fourth ramp surfaces 122 and 124 can be referred to as first and second lower distal ramp surfaces, respectively, of the first wedge member 62.

The first ramp surface 118 of the first wedge member 62 can be disposed at the first side 23 of the fusion cage 20, and the second ramp surface 120 can be disposed at the second side 25 of the fusion cage 20. Thus, the first and second ramp surfaces 118 and 120 of the first wedge member 62 can be disposed on opposite sides of the central axis 51. The third ramp surface 122 of the first wedge member 62 can be disposed at the first side 23 of the fusion cage, and the fourth ramp surface 124 can disposed at the second side 25 of the fusion cage. The helical paths of the first and second ramp surfaces 118 and 120 can be defined by a common helical pitch, but defined by different helical radii. For instance, the helical paths of the first and second ramp surfaces 118 and 120 can be defined by a single swept helix. The third and fourth ramp surfaces 122 and 124 of the first wedge member 62 can be disposed on opposite sides of the central axis 51. The helical paths of the third and fourth ramp surfaces 122 and 124 can be defined by a common helical pitch, but defined by different helical radii. For instance, the helical paths of the third and fourth ramp surfaces 122 and 124 can be defined by a single swept helix. The first ramp surface 118 and the third ramp surface 122 can be aligned with each other along the transverse direction T, and the second ramp surface 120 and the fourth ramp surface 124 can be aligned with each other along the transverse direction T. The first and second ramp surfaces 118 and 120 can face the upper endplate member 36, and the third and fourth ramp surfaces 122 and 124 can face the lower endplate member 38.

The first wedge member 62 can be movable in the expansion direction along the central axis 51. Thus, the expansion direction of the first wedge member 62 can be in a direction toward the second wedge member 64 along a curved path. As the first wedge member 62 moves in the expansion direction, the first ramp surface 118 of the first wedge member 62 rides along the first upper ramp surface 96 of the upper endplate member 36, and the second ramp surface 120 of the first wedge member 62 rides along the second upper ramp surface 98 of the upper endplate member 36. Similarly, the third ramp surface 122 of the first wedge member 62 rides along the first lower ramp surface 104 of the lower endplate member 38, and the fourth ramp surface 124 of the first wedge member 62 rides along the second lower ramp surface 106 of the lower endplate member 38. Thus, the first wedge member 62, and in particular the first and third ramp surfaces 118 and 122, urge at least one or both of the upper and lower endplate members 36 and 38 away from the other of the upper and lower endplate members 36 and 38 along the transverse direction T.

Advantageously, the first wedge member 62 can be configured to maintain surface contact with each of the upper endplate member 36 and the lower endplate member 38 when the fusion cage 20 is in the contracted position, when the fusion cage 20 is in the expanded position, and as the fusion cage 20 moves between the contracted position and the expanded position. Accordingly, each of the first and second ramp surfaces 118 and 120 of the first wedge member 62 and the third and fourth ramp surfaces 122 and 124 of the first wedge member 62 can extend along respective nonplanar paths. In one example, the first and second ramp surfaces 118 and 120 of the first wedge member 62 and the third and fourth ramp surfaces 122 and 124 of the first wedge member 62 can extend along respective twisted paths. For instance, the first and second ramp surfaces 118 and 120 of the first wedge member 62 can extend along respective helical paths. The helical paths of the first and second ramp surfaces 118 and 120 can be defined by a common helical pitch, but defined by different helical radii. For instance, the helical paths of the first and second ramp surfaces 118 and 120 can be defined by a single swept helix. The helical first and second ramp surfaces 118 and 120 of the first wedge member 62 can be complementary with the helical first and second ramp surfaces 96 and 98, respectively, of the upper endplate member 36. Thus, the helical first and second ramp surfaces 118 and 120 of the first wedge member 62 can substantially nest with the helical first and second ramp surfaces 96 and 98, respectively, of the upper endplate member 36 when the fusion cage 20 is in the contracted position, when the fusion cage 20 is in the expanded position, and as the fusion cage 20 moves between the contracted position and the expanded position.

Similarly, the third and fourth ramp surfaces 122 and 124 of the first wedge member 62 can extend along respective helical paths. The helical paths of the third and fourth ramp surfaces 122 and 124 can be defined by the same helical pitch, but defined by different helical radii. For instance, the helical paths of the third and fourth ramp surfaces 122 and 124 can be defined by a single swept helix. The helical third and fourth ramp surfaces 122 and 124 of the first wedge member 62 can be complementary with the helical first and second ramp surfaces 104 and 106, respectively, of the lower endplate member 38. Thus, the helical third and fourth ramp surfaces 122 and 124 of the first wedge member 62 can substantially nest with the helical first and second ramp surfaces 104 and 106, respectively, of the lower endplate member 38 when the fusion cage 20 is in the contracted position, when the fusion cage 20 is in the expanded position, and as the fusion cage 20 moves between the contracted position and the expanded position.

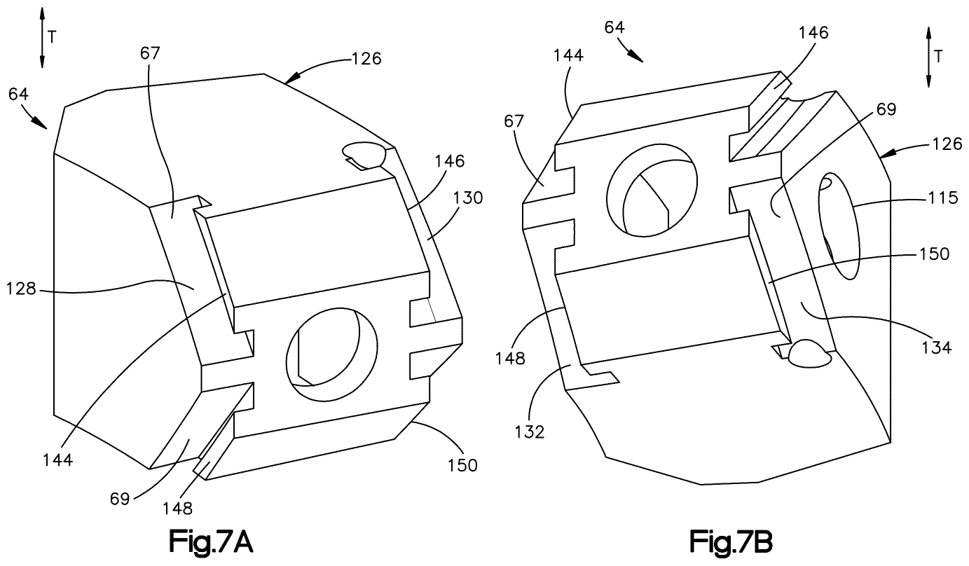

The second wedge member 64 will now be described with reference now to FIGS. 3 and 7A-7B. In particular, the second or proximal wedge member 64 has a second wedge body 126 that can be coupled to the actuator 74 of the drive assembly 68 as will be described in more detail below. The second wedge body 126, and thus the second wedge member 64, defines at least one upper proximal wedge ramp surface 67 and at least one lower proximal wedge ramp surface 69 opposite the upper proximal ramp surface 67 along the transverse direction T. The at least one upper proximal wedge ramp surface 67 of the second wedge member 64 can be sloped upward toward the upper bone contacting surface 28 as it extends in the proximal direction. The at least one lower proximal wedge ramp surface 69 of the second wedge member 64 can be sloped downward toward the lower bone contacting surface 30 as it extends in the proximal direction.

The at least one upper proximal wedge ramp surface 67 of the second or proximal wedge member 64 can include respective first and second ramp surfaces 128 and 130. The at least one lower proximal wedge ramp surface 65 of the second or proximal wedge member 64 can include respective third and fourth ramp surfaces 132 and 134, respectively. The first and second ramp surfaces 128 and 130 can be referred to as first and second upper proximal ramp surfaces, respectively, of the second wedge member 64. The third and fourth ramp surfaces 132 and 134 can be referred to as first and second lower proximal ramp surfaces, respectively, of the second wedge member 64.

The first ramp surface 128 of the second wedge member 64 can be disposed at the first side 23 of the fusion cage 20, and the second ramp surface 130 can be disposed at the second side 25 of the fusion cage 20. Thus, the first and second ramp surfaces 128 and 130 of the second wedge member 64 can be disposed on opposite sides of the central axis 51. The third ramp surface 132 of the second wedge member 64 can be disposed at the first side 23 of the fusion cage, and the fourth ramp surface 134 can disposed at the second side 25 of the fusion cage 20. Thus, the third and fourth ramp surfaces 132 and 134 of the second wedge member 64 can be disposed on opposite sides of the central axis 51. The first ramp surface 128 and the third ramp surface 132 can be aligned with each other along the transverse direction T, and the second ramp surface 130 and the fourth ramp surface 134 can be aligned with each other along the transverse direction T. The first and second ramp surfaces 128 and 130 can face the upper endplate member 36, and the third and fourth ramp surfaces 132 and 134 can face the lower endplate member 38. The helical paths of the first and second ramp surfaces 128 and 130 can be defined by a common helical pitch, but defined by different helical radii. For instance, the helical paths of the first and second ramp surfaces 128 and 130 can be defined by a single swept helix. Similarly, the helical paths of the third and fourth ramp surfaces 132 and 134 can be defined by a common helical pitch, but defined by different helical radii. For instance, the helical paths of the third and fourth ramp surfaces 132 and 134 can be defined by a single swept helix.

As will be described in more detail below, and referring also to FIGS. 8A-8D, the distal wedge member 62 can be configured to cause the upper and lower endplate members 36 and 38 to translate proximally against the proximal wedge member 64 along the curved path defined by the central axis 51. In one example, the proximal wedge member 64 can remain stationary with respect to translation along the curved central axis 51 while the endplate members 36 and 38 translate proximally against the second wedge member 64. As the upper and lower endplate members 36 and 38 translate proximally relative to the second wedge member 64 along the curved path, the third ramp surface 100 of the upper endplate member 36 rides along the first ramp surface 128 of the second wedge member 64, and the fourth ramp surface 102 of the upper endplate member 36 rides along the second ramp surface 130 of the second wedge member 64. Similarly, the third ramp surface 108 of the lower endplate member 38 rides along the third ramp surface 132 of the second wedge member 64, and the fourth ramp surface 110 of the lower endplate member 38 rides along the fourth surface 134 of the second wedge member 64.

While the proximal wedge member 64 can remain stationary as the upper and lower endplate members 36 and 38 are urged to move proximally in some examples, it should be appreciated that in other examples, the fusion cage 20 can be constructed such that the second wedge member 64 travels distally toward the first wedge member 62. Either way, it can be said that the second or proximal wedge member 64 and the upper and lower endplate members 36 and 38 translate relative to each other along the curved path. As the second wedge member 64 and the upper and lower endplate members 36 and 38 translate relative to each other along the curved path, the respective ramp surfaces of the second wedge member 64 and the ramp surfaces of the upper and lower endplate members 36 and 38 ride along each other so as to expand the fusion cage 20, or conversely to move the fusion cage 20 toward or to the contracted position. Thus, the first ramp surface 128 of the second wedge member 64 and the third ramp surface 100 of the upper endplate member 36 translate or ride along each other. Further, the second ramp surface 130 of the second wedge member 64 and the fourth ramp surface 102 of the upper endplate member 36 translate or ride along each other. Further still, the third ramp surface 132 of the second wedge member 64 and the third ramp surface 108 of the lower endplate member 38 translate or ride along each other. Further still, the fourth ramp surface 134 of the second wedge member 64 and the fourth ramp surface 110 of the lower endplate member 38 translate or ride along each other.

Advantageously, the second wedge member 64 can be configured to maintain surface contact with each of the corresponding ramp surfaces of the upper and lower endplate members 36 and 38 as the fusion cage 20 moves between the contracted position and the expanded position. Accordingly, each of the first and second ramp surfaces 128 and 130 of the second wedge member 64 and the third and fourth ramp surfaces 132 and 134 of the second wedge member 64 can extend along a respective nonlinear path. Otherwise stated, each of the first, second, third, and fourth ramp surfaces 128-134 of the second wedge member 64 can be nonplanar. In one example, the first and second ramp surfaces 128 and 130 of the second wedge member 64 and the third and fourth ramp surfaces 132 and 134 of the second wedge member 64 can extend along respective twisted paths. For instance, the first and second ramp surfaces 128 and 130 of the lower endplate member 38 can extend along respective helical paths. The helical paths of the first and second ramp surfaces 128 and 130 can be defined by a common helical pitch, but defined by different helical radii. For instance, the helical paths of the first and second ramp surfaces 128 and 130 can be defined by a single swept helix. The helical first and second ramp surfaces 128 and 130 of the second wedge member 64 can be complementary with the helical third and fourth ramp surfaces 100 and 102, respectively, of the upper endplate member 36. Thus, the helical first and second ramp surfaces 128 and 130 of the second wedge member 64 can substantially nest with the helical third and fourth ramp surfaces 128 and 130, respectively, of the upper endplate member 36 when the fusion cage 20 is in the contracted position, when the fusion cage 20 is in the expanded position, and as the fusion cage 20 moves between the contracted position and the expanded position.

Similarly, the third and fourth ramp surfaces 132 and 134 of the second wedge member 64 can extend along respective helical paths. The helical paths of the third and fourth ramp surfaces 132 and 134 can be defined by the same helical pitch, but defined by different helical radii. For instance, the helical paths of the third and fourth ramp surfaces 132 and 134 can be defined by a single swept helix. The helical third and fourth ramp surfaces 132 and 134 of the second wedge member 64 can be complementary with the helical third and fourth ramp surfaces 108 and 110, respectively, of the lower endplate member 38. Thus, the helical third and fourth ramp surfaces 132 and 134 of the second wedge member 64 can substantially nest with the helical third and fourth ramp surfaces 132 and 134, respectively, of the lower endplate member 38 when the fusion cage 20 is in the contracted position, when the fusion cage 20 is in the expanded position, and as the fusion cage 20 moves between the contracted position and the expanded position.

Referring now to FIGS. 3-7B generally, first and second wedge members 62 and 64 can each be interlinked with the upper and lower endplate members 36 and 38. That is, the first and second wedge members 62 and 64 can each include at least one protrusion that is captured by the upper endplate member 36, and at least one protrusion that is captured by the lower endplate member 38. For instance, the first wedge member 62 can include a first protrusion 136 that extends out from the first wedge body 112. The first protrusion 136 can be spaced above the first ramp surface 118, and aligned with at least a portion of the first ramp surface 118 along the transverse direction T, such that the first wedge member 62 defines a first gap that extends between the first protrusion 136 and the first ramp surface 118. The first wedge member 62 can further include a second protrusion 138 that extends out from the first wedge body 112. The second protrusion 138 can extend out from the first wedge body 112 in a direction opposite the first protrusion 136. The second protrusion 138 can be spaced above the second ramp surface 120, and aligned with at least a portion of the second ramp surface 120 along the transverse direction T, such that the first wedge member 62 defines a second gap that extends between the second protrusion 138 and the second ramp surface 120. The first and second protrusions 136 and 138 can be referred to as upper protrusions of the first wedge member 62, or an upper pair of protrusions of the first wedge member 62. The first and second protrusions 136 and 138 can be separate from each other or continuous with each other to define a single unitary protrusion as desired.

Similarly, the first wedge member 62 can include a third protrusion 140 that extends out from the first wedge body 112. The third protrusion 140 can be spaced below the third ramp surface 122, and aligned with at least a portion of the third ramp surface 122 along the transverse direction T, such that the first wedge member 62 defines a third gap that extends between the third protrusion 140 and the third ramp surface 122. The first wedge member 62 can include a fourth protrusion 142 that extends out from the first wedge body 112. The fourth protrusion 142 can be spaced below the fourth ramp surface 124, and aligned with at least a portion of the fourth ramp surface 124 along the transverse direction T, such that the first wedge member 62 defines a fourth gap that extends between the fourth protrusion 142 and the fourth ramp surface 124. The third and fourth protrusions 140 and 142 can be referred to as lower protrusions of the first wedge member 62, or a lower pair of protrusions of the first wedge member 62. The third and fourth protrusions 140 and 142 can be separate from each other or continuous with each other to define a single unitary protrusion as desired.

The second wedge member 64 can include a first protrusion 144 that extends out from the second wedge body 126. The first protrusion 144 can be spaced above the first ramp surface 128, and aligned with at least a portion of the first ramp surface 128 along the transverse direction T, such that the second wedge member 64 defines a first gap that extends between the first protrusion 144 and the first ramp surface 128. The second wedge member 64 can further include a second protrusion 146 that extends out from the second wedge body 126. The second protrusion 146 can extend out from the second wedge body 126 in a direction opposite the first protrusion 144. The second protrusion 146 can be spaced above the second ramp surface 130, and aligned with at least a portion of the second ramp surface 130 along the transverse direction T, such that the second wedge member 64 defines a second gap that extends between the second protrusion 146 and the second ramp surface 130. The first and second protrusions 144 and 146 can be referred to as upper protrusions of the second wedge member 64, or an upper pair of protrusions of the second wedge member 64. The first and second protrusions 144 and 146 can be separate from each other or continuous with each other to define a single unitary protrusion as desired.

Similarly, the second wedge member 64 can include a third protrusion 148 that extends out from the second wedge body 126. The third protrusion 148 can be spaced below the third ramp surface 132, and aligned with at least a portion of the third ramp surface 132 along the transverse direction T, such that the second wedge member 64 defines a third gap that extends between the third protrusion 148 and the third ramp surface 132. The second wedge member 64 can include a fourth protrusion 150 that extends out from the second wedge body 126. The fourth protrusion 150 can be spaced below the fourth ramp surface 134, and aligned with at least a portion of the fourth ramp surface 134 along the transverse direction T, such that the second wedge member 64 defines a fourth gap that extends between the fourth protrusion 150 and the fourth ramp surface 134. The third and fourth protrusions 148 and 150 can be referred to as lower protrusions of the second wedge member 64, or a lower pair of protrusions of the second wedge member 64. The third and fourth protrusions 148 and 150 can be separate from each other or continuous with each other to define a single unitary protrusion as desired.

As described above, the first and second wedge members 62 and 64 can be interlinked with each of the upper and lower endplate members 36 and 38. In particular, the first and second protrusions 136 and 138 of the first wedge member 62 can be disposed in respective ones of the first slots 73 of the upper endplate member 36. Similarly, the third and fourth protrusions 140 and 142 can be disposed in respective ones of the first slots 77 of the lower endplate member 38. Accordingly, the first wedge member 62 can be captured by each of the upper and lower endplate members 36 and 38. Further, as the first wedge member 62 is moved in the contraction direction, the first and second protrusions 136 and 138 can ride along respective ramped inner surfaces of the upper endplate member 36 opposite the first and second ramp surfaces 96 and 98, which can urge the upper endplate member 36 toward the lower endplate member 38 along the transverse direction T. Similarly, as the first wedge member 62 is moved in the contraction direction, the third and fourth protrusions 140 and 142 can ride along respective ramped inner surfaces of the lower endplate member 38 opposite the first and second ramp surfaces 104 and 106, which can urge the lower endplate member 48 toward the upper endplate member 36 along the transverse direction T. Thus, the height of the fusion cage 20 along the transverse direction can be reduced. Alternatively or additionally, movement of the first wedge member 62 in the contraction direction allows anatomical or other forces to cause the upper and lower endplate members 36 and 38 to move toward each other.

The first and second protrusions 144 and 146 of the second wedge member 64 can be disposed in respective ones of the second slots 75 of the upper endplate member 36. Similarly, the third and fourth protrusions 148 and 150 can be disposed in respective ones of the second slots 79 of the lower endplate member 38. Accordingly, the second wedge member 64 can be captured by each of the upper and lower endplate members 36 and 38. Further, as the first wedge member 62 is moved in the contraction direction, the first wedge member 62 urges the first and second endplate members 62 and 64 to correspondingly move in respective contraction directions, whereby the first and second protrusions 144 and 146 can ride along respective ramped inner surfaces of the upper endplate member 36 opposite the third and fourth ramp surfaces 100 and 102, which can urge the upper endplate member 36 toward the lower endplate member 38 along the transverse direction T. Similarly, the third and fourth protrusions 148 and 150 can ride along respective ramped inner surfaces of the lower endplate member 38 opposite the third and fourth ramp surfaces 108 and 110, which can urge the lower endplate member 48 toward the upper endplate member 36 along the transverse direction T. Thus, the height of the fusion cage 20 along the transverse direction T can be reduced. Alternatively or additionally, anatomical or other forces can cause the upper and lower endplate members 36 and 38 to move toward each other.

Referring now to FIGS. 3-5B, the upper and lower endplate members 36 and 38 can include respective alignment members that engage each other and guide movement of the fusion cage 20 between the contracted position and the expanded position, thereby enhancing the stability and structural integrity of the fusion cage 20.

For instance, the alignment member of the upper endplate member 36 can be supported by the first and second upper side walls 44 and 46. In one example, the alignment member of the upper endplate member 36 can include a first upper alignment slot 156 that extends into the first upper side wall 44, and a second upper alignment slot 158 that extends into the second upper side wall 46. The first and second upper alignment slots 156 and 158 can be elongate along the transverse direction T, and can extend respective along straight linear paths that are parallel to each other. In one example, the first upper alignment slot 156 extends into an inner surface of the first upper side wall 44, and a second upper alignment slot 158 that extends into an inner surface of the second upper side wall 46. The inner surfaces of the first and second side walls 44 and 46 can face each other. Alternatively, the first and second upper alignment slots 156 and 158 can extend into respective outer surfaces of the first and second side walls 44 and 46 that are opposite the inner surfaces.

The alignment member of the lower endplate member 38 can be supported by the first and second side walls 48 and 50. For instance, the alignment member of the lower endplate member 38 can include a first lower alignment rib 164 that extends out from the first lower side wall 48, and a second lower alignment rib 166 that extends out from an outer surface of the second side wall 50. For instance, the first and second lower alignment ribs 164 and 166 can extend out from respective outer surfaces of the first and second lower side walls 48 and 50 that face away from each other. In other examples, the first and second lower alignment ribs 164 and 166 can extend out from respective inner surfaces of the first and second lower side walls 48 and 50 that are opposite the outer surfaces and face each other. The alignment ribs 164 and 166 can be elongate along the transverse direction, and can thus extend respective along straight linear paths that are parallel to each other. While the alignment slots 156 and 158 are carried by the upper endplate member 36 and the ribs 164 and 166 are carried by the lower endplate member 38 in one example, it should be appreciated that the alignment slots 156 and 158 are carried by the lower endplate member 38 and the alignment ribs 164 and 166 are carried by the upper endplate member 36 as desired.

During operation, the first and second ribs 164 and 166 are disposed in the first and second slots 156 and 158, respectively, when the cage 20 is in the contracted position, when the cage 20 is in the expanded position, and as the cage 20 moves between the contracted position and the expanded position. The engagement of the ribs 164 and 166 and the slots 156 and 158 can prevent the first and second endplate members 36 and 38 from translating with respect to each other along a direction perpendicular to the transverse direction T.

The ribs 164 and 166, and the slots 156 and 158, can define any suitable shape as desired. For instance, in one example, the ribs 164 and 166 and the slots 156 and 158 can be rectangular in cross section along a plane that is perpendicular to the transverse direction T. Alternatively, the ribs 164 and 166, and the slots 156 and 158, can define interlocking dovetail shapes.

The expansion of the fusion cage 20 along the transverse direction T will now be described in more detail with initial reference to FIGS. 8A-8D. As described above, the cage 20 is movable between a contracted position and an expanded position. The upper and lower endplates 36 and 38 can abut each other when the cage 20 is in the contracted position. Thus, when the cage 20 is in the contracted position shown in FIG. 8A, the cage 20 defines a first height H1 measured along the transverse direction T from the upper bone contacting surface 28 to the lower bone contacting surface 30. Further, the upper and lower plate portions 37 and 39 can abut each other when the cage 20 is in the contracted position 20, thereby preventing the height of the cage 20 from being reduced from the first height H1. The upper and lower endplate members 36 and 38 move away from each other along the transverse direction in their respective entireties when the cage 20 is moved to the expanded position shown in FIG. 8D. When the cage 20 is in the expanded position shown in FIG. 8D, the cage 20 defines a second height H2 measured along the transverse direction T from the upper bone contacting surface 28 to the lower bone contacting surface 30 that is greater than the first height H1. Further, as will be appreciated from the description below, the first and second sides 23 and 25 of the cage 20 can expand at an equal rate. Thus, the bone contacting surfaces 28 and 30 can define a relative orientation with respect to each other that remains constant when the cage 20 is in the contracted position, when the cage 20 is in the expanded position, and as the cage 20 moves between the expanded position and the contracted position. In one example, the relative orientations of the first and second bone contacting surfaces 28 and 30 can be substantially parallel to each other. In another example, the relative orientations of the first and second bone contacting surfaces 28 and 30 can define any suitable lordotic angle.

As described above, the first side 23 of the fusion cage 20 can be concave as it extends in a direction from the distal end 32 to the proximal end 34, and the second side 25 of the fusion cage 20 can be convex as it extends in a direction from the distal end 32 to the proximal end 34. Accordingly, the ramp surfaces disposed at the first side 23 of the fusion cage 20 travel a shorter distance along the curved path than the ramp surfaces at the second side 25 of the fusion cage 20 as the fusion cage moves between the contracted and expanded positions. Conversely, the ramp surfaces disposed at the second side 25 of the fusion cage 20 travel a longer distance along the curved path than the ramp surfaces at the first side 23 of the fusion cage 20 as the fusion cage moves between the contracted and expanded positions. Accordingly, the helical ramp surfaces disposed at the first side 23 of the fusion cage 20 can define a first lead-in angle α with respect to a horizontal plane that is oriented perpendicular to the transverse direction T, and the ramp surfaces disposed at the second side 25 of the fusion cage 20 can define a second lead-in angle θ with respect to the horizontal plane that is less than the first lead-in angle. In one example, the horizontal plane can include the curved central axis 51. As a result, the relative orientation between the upper and lower endplate members 36 and 38 remain constant when the fusion cage 20 is in the contracted position, when the fusion cage 20 is in the expanded position, and as the fusion cage 20 moves between the contracted position and the expanded position. In one example, the first lead-in angle α can be in a range from approximately 20 degrees to approximately 80 degrees, such as from approximately 30 degrees to approximately 70 degrees, such as approximately 40 degrees to approximately 60 degrees, such as approximately 49 degrees with respect to the horizontal plane. The second lead-in angle θ can be in a range from approximately 20 degrees to approximately 80 degrees, such as from approximately 30 degrees to approximately 70 degrees, such as approximately 40 degrees to approximately 60 degrees, such as approximately 42 degrees with respect to the horizontal plane. It should be appreciated, of course, that the first and second lead-in angles can define any suitable respective alternative angle as desired.

For instance, as shown at FIGS. 8A-8B, each of the first and third ramp surfaces 96 and 100 of the upper endplate member 36, the first and third ramp surfaces 104 and 108 of the lower endplate member 38, the first and third ramp surfaces 118 and 122 of the first or distal wedge member 62, and the first and third ramp surfaces 128 and 132 of the second or proximal wedge member 64 can define the first lead-in angle α. Each of the first ramp surfaces 96 and 118 of the upper endplate member 36 and the first wedge member 62, respectively, and the third ramp surfaces 108 and 132 of the lower endplate member 38 and the second wedge member 64, respectively, can be defined by substantially the same pitch and substantially the same helical radius. The third ramp surface 122 of the first wedge member 62, the first ramp surface 104 of the lower endplate member 38, the third ramp surface 108 of the upper endplate member 36, and the first ramp surface 128 of the second wedge member 64 can be defined by substantially the same pitch and substantially the same helical radius. Further, the first lead-in angle α that is defined by each of the first ramp surfaces 96 and 118 of the upper endplate member 36 and the first wedge member 62, respectively, and the third ramp surfaces 108 and 132 of the lower endplate member 38 and the second wedge member 64, respectively, can be oriented opposite the first lead-in angle α that is defined by each of the third ramp surface 122 of the first wedge member 62, the first ramp surface 104 of the lower endplate member 38, the third ramp surface 108 of the upper endplate member 36, and the first ramp surface 128 of the second wedge member 64.

The first ramp surface 96 of the upper endplate member 36 and the first ramp surface 118 of the first wedge member 62 can be mirror images of the first ramp surface 104 of the lower endplate member 38 and the third ramp surface 122 of the first wedge member 62 with respect to a plane that includes the curved central axis 51. Further, the first ramp surface 96 of the upper endplate member 36 and the first ramp surface 118 of the first wedge member 62 can be mirror images of the third ramp surface 100 of the upper endplate member 36 and the first ramp surface 128 of the second wedge member 64 with respect to a plane that is oriented along the transverse direction and a direction that is perpendicular to a line tangent to the curved central axis 51. Further still, the first ramp surface 104 of the lower endplate member 38 and the third ramp surface 122 of the first wedge member 62 can be mirror images of the third ramp surface 108 of the lower endplate member 38 and the third ramp surface 132 of the second wedge member 64 with respect to a plane that is oriented along the transverse direction and a direction that is perpendicular to a line tangent to the curved central axis 51.

As shown at FIG. 8C, each of the second and fourth ramp surfaces 98 and 102 of the upper endplate member 36, the second and fourth ramp surfaces 106 and 110 of the lower endplate member 38, the second and fourth ramp surfaces 120 and 124 of the first or distal wedge member 62, and the second and fourth ramp surfaces 130 and 134 of the second or proximal wedge member 64 can define the second lead-in angle θ. The second lead-in angle θ defined by each of the second ramp surfaces 98 and 120 of the upper endplate member 36 and the first wedge member 62, respectively, and the fourth ramp surfaces 110 and 134 of the lower endplate member 38 and the second wedge member 64, respectively, can be defined by substantially the same pitch and substantially the same helical radius. The second lead-in angle θ defined by each of the fourth ramp surface 124 of the first wedge member 62, the second ramp surface 106 of the lower endplate member 38, the fourth ramp surface 102 of the upper endplate member 36, and the second ramp surface 130 of the second wedge member 64 can be defined by substantially the same pitch and substantially the same helical radius. Further, the second lead-in angle θ that is defined by each of the second ramp surfaces 98 and 120 of the upper endplate member 36 and the first wedge member 62, respectively, and the fourth ramp surfaces 110 and 134 of the lower endplate member 38 and the second wedge member 64, respectively, can be oriented opposite the fourth ramp surface 124 of the first wedge member 62, the second ramp surface 106 of the lower endplate member 38, the fourth ramp surface 102 of the upper endplate member 36, and the second ramp surface 130 of the second wedge member 64.

The second ramp surface 98 of the upper endplate member 36 and the second ramp surface 120 of the first wedge member 62 can be mirror images of the second first ramp surface 106 of the lower endplate member 38 and the fourth ramp surface 124 of the first wedge member 62 with respect to a plane that includes the curved central axis 51. Further, the second ramp surface 98 of the upper endplate member 36 and the second ramp surface 120 of the first wedge member 62 can be mirror images of the fourth ramp surface 102 of the upper endplate member 36 and the second ramp surface 130 of the second wedge member 64 with respect to a plane that is oriented along the transverse direction and a direction that is perpendicular to a line tangent to the curved central axis 51. Further still, the fourth ramp surface 102 of the upper endplate member 36 and the second ramp surface 130 of the second wedge member 64 can be mirror images of the fourth ramp surface 110 of the lower endplate member 38 and the fourth ramp surface 134 of the second wedge member 64.

Referring now to FIGS. 4A-8D generally, and as described above, the fusion cage 20 is movable from the contracted position to the expanded position, and can also be movable from the expanded position toward or to the contracted position. The first and second slopes described above allow the fusion cage 20 to move between the contracted position and the expanded position without changing a relative orientation of the upper and lower bone contacting surfaces 28 and 30, respectively. Further, as described above, the respective ramp surfaces that ride along each other can advantageously maintain surface contact with each other when the cage 20 is in the contracted position, when the cage 20 is in the expanded position, and as the cage 20 moves between the contracted position and the expanded position. That is, at least respective majorities up to respective entireties of the ramp surfaces that are in surface contact with each other can be in contact with each other when the fusion cage 20 is in the compressed position, as the fusion cage 20 iterates between the contracted position and the expanded position, and when the fusion cage 20 is in the expanded position. Thus, engagement of the ramp surfaces can maintain structural integrity while avoiding edge contact or point contact.

In particular, the first ramp surfaces 96 and 118 of the upper endplate member 36 and the first wedge member 62, respectively, can be in surface contact with each other when the fusion cage 20 is in the compressed position, as the fusion cage 20 iterates between the contracted position and the expanded position, and when the fusion cage 20 is in the expanded position. Further, the second ramp surfaces 98 and 120 of the upper endplate member 36 and the first wedge member 62, respectively, can be in surface contact with each other when the fusion cage 20 is in the compressed position, as the fusion cage 20 iterates between the contracted position and the expanded position, and when the fusion cage 20 is in the expanded position. Further, the third ramp surface 122 of the first wedge member 62 and the first ramp surface 104 of the lower endplate member 38 can be in surface contact with each other when the fusion cage 20 is in the compressed position, as the fusion cage 20 iterates between the contracted position and the expanded position, and when the fusion cage 20 is in the expanded position. Further, the fourth ramp surface 124 of the first wedge member 62 and the second ramp surface 106 of the lower endplate member 38 can be in surface contact with each other when the fusion cage 20 is in the compressed position, as the fusion cage 20 iterates between the contracted position and the expanded position, and when the fusion cage 20 is in the expanded position.

Similarly, the first ramp surface 128 of the second wedge member 64 and the third ramp surface 100 of the upper endplate member 36 can be in surface contact with each other when the fusion cage 20 is in the compressed position, as the fusion cage 20 iterates between the contracted position and the expanded position, and when the fusion cage 20 is in the expanded position. Further, the second ramp surface 130 of the second wedge member 64 and the and the fourth ramp surface 102 of the upper endplate member 36 can be in surface contact with each other when the fusion cage 20 is in the compressed position, as the fusion cage 20 iterates between the contracted position and the expanded position, and when the fusion cage 20 is in the expanded position. Further, the third ramp surface 132 of the second wedge member 64 and the third ramp surface 108 of the lower endplate member 38 can be in surface contact with each other when the fusion cage 20 is in the compressed position, as the fusion cage 20 iterates between the contracted position and the expanded position, and when the fusion cage 20 is in the expanded position. Further, the fourth ramp surface 134 of the second wedge member 64 and the fourth ramp surface 110 of the lower endplate member 38 can be in surface contact with each other when the fusion cage 20 is in the compressed position, as the fusion cage 20 iterates between the contracted position and the expanded position, and when the fusion cage 20 is in the expanded position.

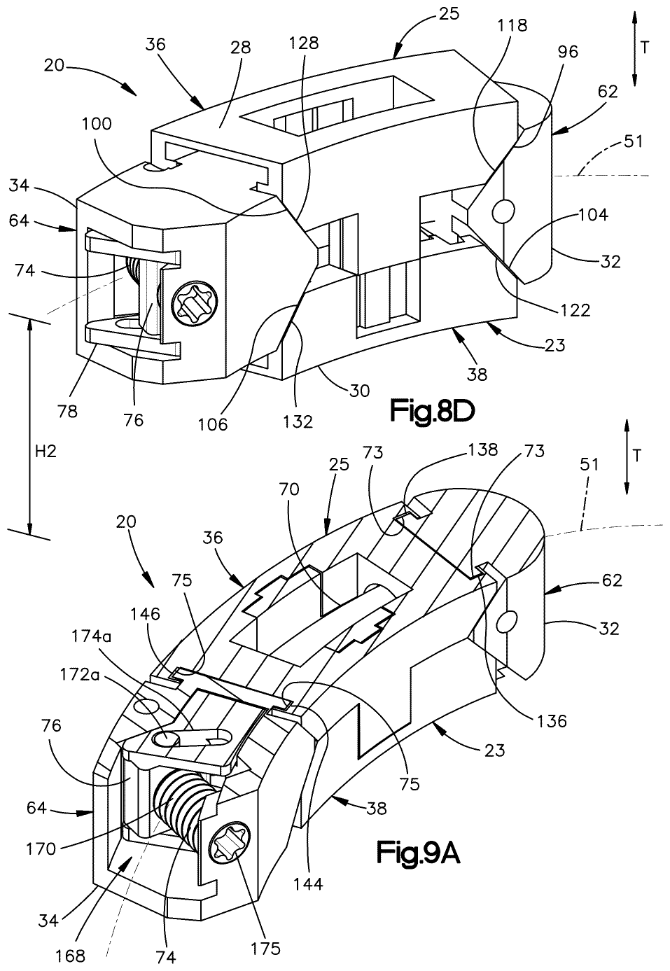

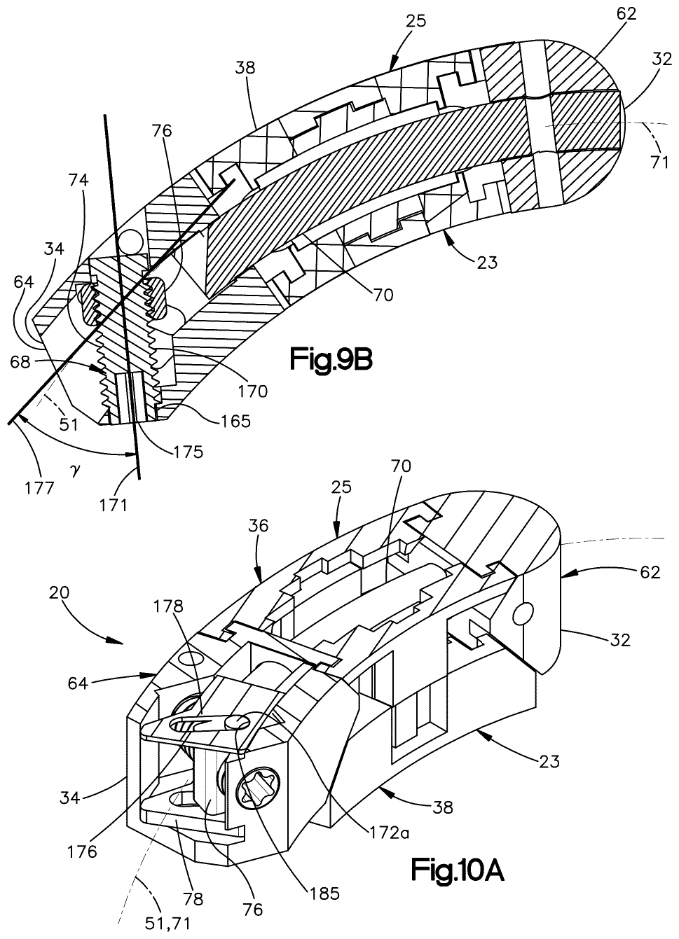

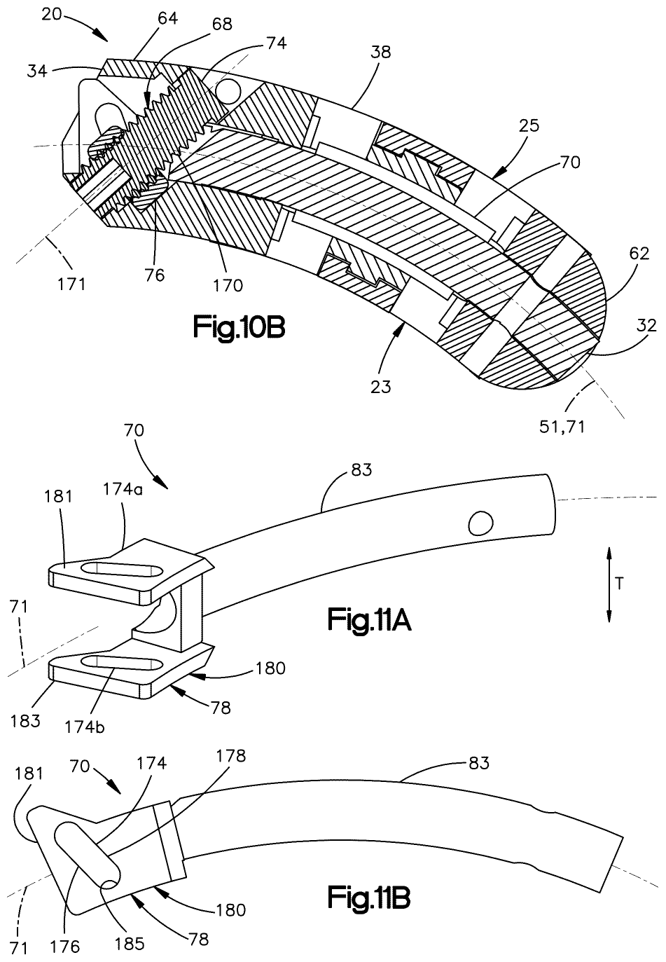

Operation of the fusion cage 20 will now be described with reference to FIGS. 3 and 9A-11B. In particular, as described above the drive assembly 68 is configured to drive the first wedge member 62 to move in the expansion direction that causes the height of the fusion cage 20 to increase. The drive assembly 68 can further be configured to drive the first wedge member 62 to move in a contraction direction that causes or allows the height of the fusion cage 20 to decrease toward or to the contracted position. As described above, the drive assembly 68 can include the drive member 70, the actuator 74, and the traveler 76, and the coupling 78. The drive assembly 68 will now be described in more detail.

The actuator 74 can be supported by any suitable support structure of the cage body 20. In one example, the actuator 74 can be supported by the proximal wedge member 64. As will be appreciated from the description below, movement of the actuator 74 in a first direction causes the distal wedge member 62 to move proximally in the expansion direction. Movement of the actuator 74 in a second direction opposite the first direction causes the distal wedge member 62 to move distally in the contraction direction. In one example, the actuator 74 can be disposed in an internal void 168 of the proximal wedge member 64. The actuator 74 can be configured as an actuator shaft 170 that is rotatably supported by the proximal wedge member 64 about an axis of rotation 171 of the actuator shaft 170. The axis of rotation 171 can define a central axis of the actuator shaft 170. For instance, at least one or both ends of the actuator shaft 170 can be supported by the proximal wedge member 64. Thus, the actuator shaft 170 is rotatable with respect to the proximal wedge member 64. Accordingly, movement of the actuator 74 in the first direction can be defined by rotation of the actuator shaft 170 with respect to the proximal wedge member 64 in a first direction of rotation about the axis of rotation 171. Conversely, movement of the actuator 74 in the first direction can be defined by rotation of the actuator shaft 170 with respect to the proximal wedge member 64 in a second direction of rotation about the axis of rotation 171 that is opposite the first direction of rotation.

The actuator shaft 170 can define any suitable drive mechanism 175, which can be configured as a socket or projection that is configured to be engaged by a drive instrument that, in turn, applies a torsional force to the actuator shaft 170 that is configured to drive the actuator to rotate about the axis of rotation 171 selectively in the first direction of rotation and the second direction of rotation. The proximal wedge member 64 can define an access aperture 165 that exposes the drive mechanism 175. The drive mechanism 175 can be advantageously positioned and oriented for easy access after the fusion cage has been inserted into the intervertebral space. In one example, the axis of rotation 171 can intersect the central axis 51 of the fusion cage 20 at an intersection point. The axis of rotation 171 can thus define an angle γ with respect to a line 177 that is tangent to the central axis 51 at the intersection point. The angle can be in a range from approximately 25 degrees to approximately 60 degrees, such as from approximately 35 degrees to approximately 50 degrees, for instance approximately 47 degrees. In other examples, the transverse direction T and the axis of rotation 171 can define a first plane that intersects the central axis 51 at a point of intersection, and the first plane can define an angle with a second plane that is tangent to the central axis 51 at the point of intersection. The angle defined by the second planes can be in a range from approximately 25 degrees to approximately 60 degrees, such as from approximately 35 degrees to approximately 50 degrees, for instance approximately 47 degrees. The actuator shaft 170 can be positionally fixed, such that the angles described above can be fixed angles. As a result, in some examples the actuator shaft 170 is easily accessible to the surgeon without requiring positional manipulation of the actuator shaft.

The traveler 76 can be supported by the actuator shaft 170, and is configured to travel along the actuator shaft 170. For instance, as described in more detail below, the traveler 76 is configured to travel along the actuator shaft 170 in a first direction of travel that causes the distal wedge member 62 to move proximally in the expansion direction. The traveler 76 can also be configured to travel along the actuator shaft 170 in a second direction of travel opposite the first direction of travel that causes the distal wedge member 62 to move distally in the contraction direction. In one example, the actuator shaft 170 and the traveler 76 can be threaded, and threadedly coupled to each other. For instance, the traveler 76 can include a traveler body 173 that defines a threaded bore 179 that threadedly receives the threaded actuator shaft 170. Thus, rotation of the actuator shaft 170 in the first direction of rotation causes the traveler 76 to travel along the actuator shaft 170 in the first direction of travel. Rotation of the actuator shaft 170 in the second direction of rotation causes the traveler 76 to travel along the actuator shaft 170 in the second direction of travel.

The coupling 78 can be movably coupled to the traveler 76, such that movement of the traveler in the first direction correspondingly causes the coupling 78 to move in a respective first direction. Conversely, movement of the traveler 76 in the second direction causes the coupling 78 to move in a respective second direction that is opposite the respective first direction of the coupling 78. Movement of the coupling 78 in the respective first direction causes the drive member 70 to move proximally in the expansion direction. The traveler 76 can include at least one actuation post 172 that rides in a corresponding at least one an oblique slot 174 of the coupling 78. The at least one actuation post 172 can be monolithic with the traveler 76, or can be attached to the traveler 76 as desired. The oblique slot 174 can be oblique to the central axis 71 of the drive member 70. In one example, the oblique slot 174 extends along a respective central axis that defines a first plane with the transverse direction T. The first plane intersects the central axis 71 of the drive member 70 at a point of intersection, and the first plane is oblique to a tangent plane that is defined by the transverse direction T and a line tangent to the central axis 71 at the point of intersection. The central axis of the oblique slot 174 can also be oblique to the axis of rotation 171 of the actuator shaft 170. That is, a first plane that includes the oblique slot 174 intersects a second plane that includes the axis of rotation 171 of the actuator shaft 170. The first and second planes can also include the transverse direction T. As the traveler 76 translates along the actuator shaft 170, the actuator post 172 rides along the slot 174, which causes the coupling 78 to urge the drive member 70 to cause the distal wedge member 62 to move selectively in the expansion direction and the contraction direction.

The oblique slot 174 is defined by a first and second opposed oblique major surfaces 176 and 178. The first major surface 176 can be referred to as an expansion surface. In this regard, when the traveler 76 moves in the respective first direction, the actuation post 172 bears against the first major surface 176, which causes the coupling 78 to urge the drive member 70, and thus the distal wedge member 62, to translate proximally in the expansion direction. Conversely, the second major surface 178 can be referred to as a contraction surface. In this regard, when the traveler 76 moves in the respective second direction, the actuation post 172 bears against the second major surface 178, which causes the coupling 78 to urge the drive member 70, and thus the distal wedge member 62, to translate distally in the contraction direction.

The coupling 78 can include a coupling body 180 and the at least one oblique slot 174 that extends at least into or through the coupling body 180 along the transverse direction T. In one example, the coupling body 180 can define a first or upper oblique slot 174a and a second or lower oblique slot 174b. Similarly, the traveler 76 can include a first or upper actuation post 172a and a second or lower actuation post 172b (see FIG. 3). The upper actuation post 172a can be upwardly extending from the traveler body 173 of the traveler 76, and the lower actuation post 172b can be downwardly extending from the traveler body 173. The upper actuation post 172a extends into the upper oblique slot 174a, and the lower actuation post 172b extends into the lower oblique slot 174b. In one example, the coupling body 180 can include a first or upper plate 181 and a second or lower plate 183 opposite the upper plate along the transverse direction T. The upper oblique slot 174a can extend into or through the upper plate 181 along the transverse direction T, and the lower oblique slot 174a can extend into or through the lower plate 181 along the transverse direction T. The traveler body 173 can be disposed between the upper and lower plates 181 and 183. The upper oblique slot 174a can be parallel with, such as aligned with, the lower oblique slot 174b, or otherwise configured as desired. The coupling 78 can be monolithic with the shaft 83 of the drive member 70 so as to define a single unitary structure. Alternatively, the coupling 78 can be separate from the shaft 83 and attached to the shaft 83.

As described above, movement of the traveler 76 in the respective first direction of travel causes the coupling to move the drive member 70, and thus the first wedge member 62 that is coupled to the drive member 70, proximally in the direction of expansion. As the distal wedge member 62 moves proximally, the distal wedge member 62 abuts the upper and lower endplate members 36 and 38, and urges the upper and lower endplates 36 and 38 to translate proximally against the proximal wedge member 64.

Once the upper and lower endplate members 36 and 38 are in contact with each of the distal and proximal wedge members 62 and 64 further movement of the distal wedge member 62 in the expansion direction causes i) the first and second ramp surfaces 96 and 98 of the upper endplate member 36 to ride along the first and second ramp surfaces 118 and 120, respectively, of the distal wedge member 62, and ii) the third and fourth ramp surfaces 100 and 102 of the upper endplate member 36 to ride along the first and second ramp surfaces 128 and 130, respectively, of the proximal wedge member 64, thereby causing the upper endplate member 36 to move away from the lower endplate member 38 along the transverse direction T. Thus, the distal wedge member 62 and the proximal wedge member 64 urge the upper endplate member 36 away from the lower endplate member 38 along the transverse direction T.

Further, once the upper and lower endplate members 36 and 38 are in contact with each of the distal and proximal wedge members 62 and 64 further movement of the distal wedge member 62 in the expansion direction causes i) the first and second ramp surfaces 104 and 106 of the lower endplate member 38 to ride along the third and fourth ramp surfaces 122 and 124, respectively, of the distal wedge member 62, and ii) the third and fourth ramp surfaces 108 and 110 of the lower endplate member 38 to ride along the third and fourth ramp surfaces 132 and 134, respectively, of the proximal wedge member 64, thereby causing the lower endplate member 38 to move away from the upper endplate member 36 along the transverse direction T. Thus, the distal wedge member 62 and the proximal wedge member 64 urge the lower endplate member 38 away from the upper endplate member 36 along the transverse direction T.

It is recognized of course, that in certain surgical applications, the lower endplate member 38 can be supported, for instance by the inferior vertebral surface 42 of the inferior vertebral body 26 (see FIGS. 1-2). Accordingly, when the fusion cage 20 is expanded from the contracted position to the expanded position, the lower endplate member 38 can remain supported by the inferior vertebral surface, such that upper endplate member 36 moves away from the lower endplate member 38 while the lower endplate member 38 remains substantially stationary with respect to expansion along the transverse direction T. However, as this is due to the particular surgical application, it can nevertheless be said that the upper and lower endplate members 36 and 38 are movable away from each other along the transverse direction T as described above. In other embodiments, the wedge members 62 and 64 can include only their upper ramp surfaces and do not include lower ramp surfaces, such that the wedge members 62 and 64 urge the upper endplate member 36 away from the lower endplate member 38 without urging the lower endplate member 38 away from the upper endplate member 36 as the fusion cage 20 moves to the expanded position.

The fusion cage 20 can include a stop member 185 that can be configured to limit expansion of the fusion cage 20. In particular, oblique slot 174 can define a terminal end. The traveler 76 is configured to move in the first direction of travel until the actuation post 172 is positioned at the terminal end of the oblique slot 174, at which point the actuation post 172 abuts the traveler body 173, which defines a stop surface that prevents further translation of the traveler 76 in the first direction of travel. Further, the actuator shaft 170 is unable to be further rotated in the first direction of rotation when the actuation post 172 is in abutment with the traveler body 173 at the terminal end of the oblique slot 174. Accordingly, abutment of the traveler post 172 with the traveler body 173 at the terminal end of the oblique slot 174 prevents further expansion of the fusion cage 20. The stop member can therefore prevent overexpansion of the fusion cage 20.

As described above, the fusion cage 20 is movable from the expanded position toward or to the contracted position, for instance when it is desired to reduce the height of the intervertebral fusion cage 20. In particular, the actuator 74 is rotated in the second direction of rotation about the axis of rotation 171, as described above, which causes the traveler 76 to translate along the actuator 74 in the second direction of travel. It should be appreciated that the traveler 76 can translate along the actuator 74 in the first and second directions of travel without rotating along the axis of rotation 171. As the traveler 76 translates along the actuator 74 in the second direction of travel, the actuation posts 172 bear against the first major surfaces 176 of the slots 174, which causes the coupling 78 to urge the drive member 70, and thus the distal wedge member 62, to translate distally in the contraction direction. The upper and lower endplates 36 and 38 can move distally toward the distal ramp 62 as the fusion cage 20 moves from the expanded position toward and to the contracted position.

As the distal wedge member 62 translates distally, the first and second protrusions 136 and 138 of the distal wedge member 62 bear against the distal end of the upper endplate member 36 and urge the distal end of the upper endplate member 36 to translate toward the lower endplate member 38 along the transverse direction T, and the third and fourth protrusions 140 and 142 of the distal wedge member 62 urge the distal end of the lower endplate 38 to translate toward the upper endplate member 36 along the transverse direction T. Similarly, the first and second protrusions 144 and 146 of the proximal wedge member 64 bear against the proximal end of the upper endplate member 36 and urge the proximal end of the upper endplate member 36 to translate toward the lower endplate member 38 along the transverse direction T, and the third and fourth projections 148 and 150 of the proximal wedge member 64 bear against the proximal end of the lower endplate member 38 and urge the proximal end of the lower endplate member 38 to translate toward the upper endplate member 36 along the transverse direction T

It is appreciated that the fusion cage 20 can define the expanded position when the distal wedge member 62 is disposed at any position spaced in the expansion direction from the contracted position. The fusion cage 20 is configured to maintain the expanded position in response to compressive anatomical forces applied by the inferior vertebral body 26 and the superior vertebral body 24, while the ramp surfaces are in abutment with each other. In one example, frictional forces between the ramp surfaces, and further between the actuator shaft 170 and the proximal wedge member 64 can prevent the compressive anatomical forces from causing the proximal wedge member 64 to move in the contraction direction.

Methods associated with the fabrication and use of the fusion cage 20 are also envisioned as described above. For instance, during use, the fusion cage 20 can be inserted into the intervertebral space along any suitable approach as desired, such as a transforaminal approach. The fusion cage 20 can be inserted in its contracted position. Alternatively, if desired, the fusion cage 20 can be inserted in an expanded position that is expanded relative to the contracted position, but not fully expanded. After the fusion cage 20 has been inserted, the fusion cage 20 can be expanded in the manner described above. If it is desired to adjust the position of the fusion cage 20 or remove the fusion cage 20, the fusion cage can be contracted toward or to the contracted position. Bone graft material can be introduced into the fusion cage 20 prior to implantation of the fusion cage 20 in the disc space. The bone graft material can extend through the respective bone graft apertures to assist with fusion of the fusion cage 20 to the vertebral bodies after implantation of the fusion cage 20.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. An expandable intervertebral fusion cage for insertion in an intervertebral space defined between a superior vertebral body and an inferior vertebral body, the fusion cage comprising:
an implant body configured to be inserted between an inferior vertebral body and a superior vertebral body;
a drive member that extends along a curved path; and
a wedge member is supported by the drive member,
wherein the drive member is configured to translate along the curved path without relative rotation between the wedge member and the drive member, which causes the wedge member to vertically expand the implant body.

2. The expandable intervertebral fusion cage of claim 1, wherein the drive member is rigid.

3. The expandable intervertebral fusion cage of claim 1, wherein drive member is curved along a plane that is oriented perpendicular to the vertical expansion.

4. The expandable intervertebral fusion cage of claim 1, wherein the wedge member is a distal wedge member that has a distal wedge ramp surface that rides along the implant body to cause the implant body to vertically expand, the expandable fusion cage further comprising:
a proximal wedge member having a proximal wedge ramp surface, wherein movement of the distal wedge member in the expansion direction drives the implant body against the proximal wedge member, such that the proximal wedge ramp surface urges a proximal end of the implant body to vertically expand.

5. An expandable intervertebral fusion cage for insertion in an intervertebral space defined between a superior vertebral body and an inferior vertebral body, the fusion cage comprising:
an upper endplate member defining an upper bone contacting surface configured to abut the superior vertebral body;
a lower endplate member defining a lower bone contacting surface configured to abut the inferior vertebral body, wherein the upper and lower bone contacting surfaces are opposite each other along a transverse direction; and
a wedge member having a helical ramp surface that is configured to urge at least one of the upper and lower endplate members away from the other of the upper and lower endplate members along the transverse direction.

6. The expandable intervertebral fusion cage of claim 5, wherein the wedge member is configured to travel in an expansion direction that urges the at least one of the upper and lower endplate members away from the other of the upper and lower endplate members along the transverse direction, and the wedge member is configured to travel in the expansion direction without relative rotation between the wedge member and the drive member.

7. A method of implanting an expandable intervertebral fusion cage, the method comprising the steps of:
inserting an implant body between an inferior vertebral body and a superior vertebral body;
translating a drive member along a curved path, such that a wedge member supported by the drive member translate with the drive member along the curved path without relative rotation between the wedge member and the drive member, which causes the wedge member to vertically expand the implant body.

8. The method of claim 7, wherein the drive member is rigid.

9. The method of claim 7, wherein drive member is curved along a plane that is oriented perpendicular to the vertical expansion.

10. The method of claim 7, wherein the wedge member is a distal wedge member that has a distal wedge ramp surface that rides along the implant body to cause the implant body to vertically expand, the expandable fusion cage further comprising:
a proximal wedge member having a proximal wedge ramp surface, wherein movement of the distal wedge member in the expansion direction drives the implant body against the proximal wedge member, such that the proximal wedge ramp surface urges a proximal end of the implant body to vertically expand.

11. The method of claim 7, wherein the wedge member defines a helical ramp surface that bears against the implant body so as to cause the implant body to vertically expand during the translating step.
